(19) United States
(12) Patent Application Publication
(10) Pub. No.: US 2007/0276024 A1
(43) Pub. Date: Nov. 29, 2007

(54) Methods for Treating Diseases and Conditions with Inverse Agonists and for Screening for Agents Acting as Inverse Agonists

(75) Inventor: Richard A. Bond, Houston, TX (US)

Correspondence Address: CATALYST LAW GROUP, APC, 9710 SCRANTON ROAD, SUITE S-170, SAN DIEGO, CA, 92121, US

(73) Assignee: Inverseon , Inc., San Francisco, CA (US)

(21) Appl. No.: 10/574,429

(22) PCT Filed: Oct. 8, 2004
(86) PCT No.: PCT/US04/33530

§ 371 Date: Feb. 1, 2007

Related U.S. Application Data
(60) Provisional application No. 60/510,250, filed on Oct. 10, 2003.
(60) Provisional application No. 60/555,797, filed on Mar. 23, 2004.

(51) Int. Cl. A61K 31/135; A61K 31/18; A61K 31/40; A61P 43/00; C12Q 1/02; G01N 33/00

(52) U.S. Cl. 514/411; 435/29; 436/501; 514/604; 514/656; 514/657

(57) ABSTRACT

The present invention describes a method for treating a disease or condition associated with the activity of a G protein coupled receptor (GPCR) comprising administering an inverse agonist for the GPCR, alone or in combination with an agonist for the GPCR, to an organism with a disease or condition associated with the activity of the GPCR in a quantity and for a period that causes an increase in the population of spontaneously active GPCRs associated with that physiological function, thereby producing a therapeutic effect to ameliorate the disease or condition. This provides a basis for so-called “paradoxical pharmacology.” These methods can be used to treat pulmonary airway diseases, including asthma and chronic allergic rhinitis, among other diseases and conditions, including obesity. The present invention further describes a screening method for screening a compound for inverse agonist activity to a GPCR.

### CROSS-REFERENCES

This application claims priority from Provisional Application Ser. No. 60/510,250, by Richard A. Bond, entitled “Method of Treating Airway Diseases with Beta-Adrenergic Agonists,” filed Oct. 9, 2003, which is incorporated herein in its entirety by this reference. This application also claims priority from Provisional Application Ser. No. 60/555,797 by Richard A. Bond, entitled “Methods for Treating Diseases and Conditions with Inverse Agonists and for Screening for Agents Acting as Inverse Agonists,” filed Mar. 23, 2004, which is also incorporated herein in its entirety by this reference.

### STATEMENT REGARDING FEDERAL FUNDED RESEARCH

Certain of the research leading to the invention recited in this application has been funded by grants from the National Institutes of Health. The United States government may therefore have certain rights in this invention.

### BACKGROUND OF THE INVENTION

This invention is directed to methods for treating chronic diseases and conditions with inverse agonists, particularly with inverse agonists for G protein coupled receptors, and to methods for screening for agents capable of acting as inverse agonists, particularly as inverse agonists for G protein coupled receptors.

A large number of diseases and conditions are related to the activities of G protein coupled receptors (GPCR). The superfamily of G protein coupled receptors includes a large number of receptors. These receptors are integral membrane proteins characterized by amino acid sequences that contain seven hydrophobic domains, predicted to represent the transmembrane spanning regions of the proteins. They are found in a wide range of organisms and are involved in the transmission of signals to the interior of cells as a result of their interaction with heterotrimeric G proteins. They respond to a diverse range of agents including lipid analogues, amino acid derivatives, small molecules such as epinephrine and dopamine, and various sensory stimuli. The properties of many known GPCR are summarized in S. Watson & S. Arkinstall, “The G-Protein Linked Receptor Facts Book” (Academic Press, London, 1994), incorporated herein by this reference.

Drug discovery for GPCRs has focused on identifying agonists or antagonists. These drugs have been traditionally characterized by their acute effects on the GPCR. Agonists are “activators” whereas antagonists are “inactivators” or “blockers”. A new understanding of GPCR function has led to the reclassification of antagonists into two subclasses, those that are competitive to agonists and exhibit partial agonist activity and those that are inverse agonists. This reclassification is based on the “two-state” model of GPCR function (Lefkowitz reference) in which the GPCR spontaneously alternates from an active state to an inactive state in the absence of a bound ligand. Based on this model, an agonist is now generally defined as a compound that stabilizes the active state of the GPCR whereas the inverse agonist stabilizes the inactive state of the GPCR.

Another consequence of this two-state model of GPCRs is the presence of a low level of spontaneous activity of the GPCR signal transduction pathway even in the absence of agonist. This was demonstrated in vivo by the inventor in mice in which transgenic mice were generated overexpressing the β₂ adrenergic receptor in heart myocardium. These mice exhibited elevated heart rates as if they were supplemented with large doses of agonist despite the fact that they were not.

Among the diseases and conditions associated with the activity of GPCR are asthma and other obstructive respiratory diseases and congestive heart failure (CHF), obesity, dementias including Alzheimer's Disease, and Parkinson's Disease. Other disorders are associated with hypofunction of the GPCR pathway but it is unclear if this is causal in the disease or not. These disorders include the desensitization of adrenergic signaling in Type I Diabetes that can lead to a condition in which the normally occurring physiological warning signals that are associated with low blood sugar are absent. This condition has been designated “hypoglycemia unawareness.” These disorders have in common hypofunction of the GPCR signaling process which, whilst not wanting to be held to theory, may be due to a reduced level of the GPCR protein itself, internal signaling components such as cyclic AMP (cAMP) which act as a “second messenger,” or increased levels of components that activate undesired signaling within the cells, such as phospholipase C.

Even though the acute effects of inverse agonists is to reduce the activity of GPCR below baseline, the chronic effects of inverse agonists may be diametrically opposite, resulting in increased activity of the GPCRs. This may be due to several effects working alone or in concert: increase in receptor numbers due to receptor stabilization, increase in internal coupling components such as Gₛα, or reduction in undesired signal-activating components such as phospholipase C. Thus, chronic treatment with inverse agonists may enhance signaling and counteract the desensitizing effects of chronic agonist treatment.

There are a large number of diseases, syndromes, and conditions for which conventional treatment is the use of agonists specific for GPCR. However, the limitations of this conventional treatment have become apparent. As indicated above, chronic administration of agonists can lead to desensitization and depression of receptor signaling. This limits the effectiveness of treatment over time. There is therefore a need for a new, global, strategy of treating diseases, syndromes, and conditions that are characterized by the hypofunction of GPCR.

These conditions include, besides pulmonary airway disorders such as asthma, conditions characterized by hypofunction of muscarinic receptors such as Alzheimer's disease. They also include chronic pain, which is frequently treated by chronic agonist treatment, which leads to tolerance and a need for an increasing dose of agonist. As indicated above, a large number of these conditions exist.

Additional physiological activities, such as the sense of smell, are also affected by the activity of GPCR. This includes the sense of smell, which, is also characterized by tolerance or reduction of signaling after chronic exposure to the same odor. This has often been considered psychological, and called accommodation, but may also have a basis in receptor biochemistry.

One class of conditions affected by GPCR hypofunction is pulmonary airway diseases, including asthma, chronic obstructive pulmonary disease (COPD), and other similar diseases and conditions. Asthma itself is of increasing concern. The incidence of asthma is increasing rapidly, particularly in children living in inner-city environments. The reasons for this increase are not clear, but various causes have been suggested, including dust mites, automobile-related pollution, and exposure to tobacco smoke. This disease is causing increasing morbidity and even mortality in many communities.

Patients with asthma and other airway disorders may have airway spasms, further reducing airflow through the pulmonary tree. During an attack, a patient's airway is constricted leading to difficulty breathing. Airway smooth muscle is responsible for the bronchoconstriction. The airway smooth muscle cells express β₂ adrenergic receptors. Agonist binding to these receptors, such as epinephrine or β₂ agonist drugs results in smooth muscle relaxation.

Consequently, for acute bronchospasms many patients inhale short-acting β₂ adrenergic agonists which function to immediately relax smooth muscle of the airway. Alternatively, asthmatics may take long-acting β₂ adrenergic agonists to prevent or reduce the severity of asthma attacks.

However, chronic administration of β-adrenergic agonists has been demonstrated to lead to drug tolerance. Furthermore, there is also an increased hyperresponsiveness of the pulmonary airway in response to provocation such as allergens.

Epidemiological studies have demonstrated a positive correlation between the chronic use of short-acting β-adrenergic agonists and asthma mortality. A large trial with the long-acting β₂-adrenergic agonist, salmeterol, was stopped due to increased death rates. This underscores that while short-term administration of β-agonists may be helpful to asthmatic patients, long-term administration may be deleterious.

Consequently, there is tremendous need for new therapeutic alternatives to β agonist use in asthmatics. There is also a substantial need for new therapeutic alternatives for treating CHF and other diseases and conditions associated with GPCR.

### SUMMARY OF THE INVENTION

One aspect of the present invention is a method for treating a disease or condition associated with the activity of a G protein coupled receptor (GPCR) comprising administering an inverse agonist for the GPCR to an organism with a disease or condition associated with the activity of the GPCR in a quantity and for a period that causes an increase in the population of GPCRs, either spontaneously active, those that are available and activated by an endogenous agonist or by an exogenous agonist, associated with that physiological function, thereby producing a therapeutic effect to ameliorate the disease or condition. Another aspect of the invention is administering an inverse agonist for the GPCR to an organism with a disease or condition associated with the activity of the GPCR in a quantity and for a period that prevents the decrease in the population of GPCRs due to the presence of either exogenous or endogenous agonist.

Typically, the administration of the inverse agonist results in continuous levels of the inverse agonist in the bloodstream of the organism to which the inverse agonist is being administered.

The disease or condition can be a pulmonary airway disease, such as asthma, allergic rhinitis, bronchiectasis, bronchitis, chronic obstructive pulmonary disease (COPD), Churg-Strauss syndrome, the pulmonary sequelae of cystic fibrosis, emphysema, or pneumonia.

When the disease or condition is a pulmonary airway disease, the therapeutic effect is typically a reduction in pulmonary airway constriction hyperresponsiveness. When the disease or condition is a pulmonary airway disease, typically the GPCR is a β₂-adrenergic receptor, and the therapeutic effect is an upregulation of the population of these receptors. When the disease or condition is a pulmonary airway disease, the inverse agonist can be selected from the group consisting of nadolol, bupranolol, butoxamine, carazolol, carvedilol, ICI-118,551, levobunolol, metoprolol, propranolol, sotalol, and timolol, and the salts, solvates, analogues, congeners, bioisosteres, hydrolysis products, metabolites, precursors, and prodrugs thereof. Typically, the inverse agonist is nadolol or carvedilol.

The method can further comprise the administration of an additional agent, such as a β₂-selective adrenergic agonist drug, a steroid, an anticholinergic drug, a xanthine compound, an anti-IgE antibody, a leukotriene modifier, or a phosphodiesterase IV inhibitor.

Alternatively, the disease or condition can be congestive heart failure.

In yet another alternative, the disease or condition can be associated with acetylcholine receptors, α-adrenergic receptors, β₃-adrenergic receptors, serotonin (5-hydroxytryptamine) receptors, dopamine receptors, adenosine receptors, angiotensin Type II receptors, bradykinin receptors, calcitonin receptors, calcitonin gene-related receptors, cannabinoid receptors, cholecystokinin receptors, chemokine receptors, cytokine receptors, gastrin receptors, endothelin receptors, γ-aminobutyric acid (GABA) receptors, galanin receptors, glucagon receptors, glutamate receptors, luteinizing hormone receptors, choriogonadotrophin receptors, follicle-stimulating hormone receptors, thyroid-stimulating hormone receptors, gonadotrophin-releasing hormone receptors, leukotriene receptors, Neuropeptide Y receptors, opioid receptors, parathyroid hormone receptors, platelet activating factor receptors, prostanoid (prostaglandin) receptors, somatostatin receptors, thyrotropin-releasing hormone receptors, vasopressin and oxytocin receptors.

The method can further comprise the administration of an agonist to the GPCR along with the inverse agonist.

Another aspect of the invention is a method for screening a compound for inverse agonist activity against a GCPR comprising the steps of:

(1) providing a population of specific G protein coupled receptors characterized by a constitutive basal level of activity in the absence of an agonist;

(2) contacting the population of specific G protein coupled receptors with a compound to be screened for its inverse agonist activity, the compound not being an agonist of the population of specific G protein coupled receptors; and

(3) determining the constitutive basal level of activity of the specific G protein coupled receptors in the absence of the compound and in the presence of the compound, such that the constitutive basal level of activity decreases if the compound is an inverse agonist.

Yet another aspect of the invention is a method for screening a compound for inverse agonist activity against a GCPR comprising the steps of:

(1) providing cells containing a population of specific G protein coupled receptors characterized by a constitutive basal level of activity in the absence of an agonist;

(2) contacting the cells containing the population of specific G protein coupled receptors with a compound to be screened for its inverse agonist activity, the compound not being an agonist of the population of specific G protein coupled receptors, the compound being contacted with the cells for a period of time to result in an increase in receptor population or receptor density if the compound is an inverse agonist; and

(3) determining the receptor population or receptor density of the specific G protein coupled receptors in the cells in the absence of the compound and in the presence of the compound, such that the receptor population or receptor density increases if the compound is an inverse agonist.

Another aspect of the invention is a blister pack comprising:

(1) a lower substrate;

(2) an intermediate dosage holder that is shaped to generate a plurality of cavities and that is placed over the lower substrate, the cavities being shaped to hold dosage forms of an inverse agonist for a GPCR;

(3) an upper substrate placed over the intermediate dosage holder that has a plurality of apertures, each aperture being located to accommodate a corresponding cavity; wherein the dosage forms are of graduated dosages starting with a lowest dose and proceeding to a highest dose; and

(4) dosage forms of the inverse agonist for the GPCR placed in the cavities.

Yet another aspect of the invention is a pharmaceutical composition comprising:

(1) a therapeutically effective amount of an inverse agonist for a GPCR;

(2) a therapeutically effective amount of a second therapeutic agent, the second therapeutic agent being selected from the group consisting of a β₂-selective adrenergic agonist, a steroid, an anticholinergic drug, a xanthine compound, an anti-IgE antibody, a leukotriene modifier, and a phosphodiesterase IV inhibitor; and

(3) a pharmaceutically acceptable carrier.

Yet another aspect of the invention is a blister pack comprising:

(1) a lower substrate;

(2) an intermediate dosage holder that is shaped to generate a plurality of cavities and that is placed over the lower substrate, the cavities being shaped to hold dosage forms of the pharmaceutical composition comprising an inverse agonist for a GPCR and a second therapeutic agent;

(3) an upper substrate placed over the intermediate dosage holder that has a plurality of apertures, each aperture being located to accommodate a corresponding cavity; and

(4) dosage forms of the pharmaceutical composition placed in the cavities.

Yet another aspect of the invention is a blister pack comprising:

(1) a lower substrate;

(2) an intermediate dosage holder that is shaped to generate a plurality of cavities and that is placed over the lower substrate, the cavities being shaped to hold dosage forms of: (a) a first pharmaceutical composition that comprises: (i) a therapeutically effective amount of an inverse agonist for a GPCR; and (ii) a first pharmaceutically acceptable carrier; and (b) a second pharmaceutical composition that comprises: (i) a therapeutically effective amount of a second therapeutic agent, the second therapeutic agent being selected from the group consisting of a β₂-selective adrenergic agonist, a steroid, an anticholinergic drug, a xanthine compound, an anti-IgE antibody, a leukotriene modifier, and a phosphodiesterase IV inhibitor; and (ii) a second pharmaceutically acceptable carrier;

(3) an upper substrate placed over the intermediate dosage holder that has a plurality of apertures, each aperture being located to accommodate a corresponding cavity; and

(4) dosage forms of the first and second pharmaceutical compositions placed in the cavities.

### BRIEF DESCRIPTION OF THE DRAWINGS

These and other features, aspects, and advantages of the present invention will become better understood with reference to the following description, appended claims, and accompanying drawings where:

FIG. 1 is a diagram of a blister pack holding dosage forms of inverse agonists according to the invention.

FIG. 2A is a graph showing that methacholine provocation significantly enhances airway resistance (R_{aw}) in asthmatic mice.

FIG. 2B is a similar graph showing that saline provocation, as a control, does not significantly enhance airway resistance in asthmatic mice.

FIG. 2C is a similar graph showing that the administration of a single intravenous bolus of salbutamol to asthmatic mice reduced the level of airway responsiveness to methacholine provocation and the level of airway resistance.

FIG. 2D is a similar graph showing that no protection was observed when salbutamol was delivered to the mice for 28 days before methacholine provocation.

FIG. 2E is a similar graph showing that when asthmatic mice were given a single intravenous bolus of alprenolol, a β-adrenergic antagonist with partial agonist activity, their airway responsiveness was diminished.

FIG. 2F is a similar graph showing that when asthmatic mice were exposed to alprenolol for 28 days, their average methacholine dose-response relationship was similar to that obtained in nontreated mice, demonstrating that this drug provides no benefit upon chronic administration.

FIG. 2G is a similar graph showing that a single intravenous bolus of carvedilol enhanced the airway responsiveness in the asthmatic mice.

FIG. 2H is a similar graph showing that chronic administration of carvedilol reduced the responsiveness of asthmatic mice to methacholine provocation.

FIG. 21 is a similar graph showing that a single intravenous bolus of nadolol also enhanced the airway responsiveness of asthmatic mice similar to that observed for carvedilol.

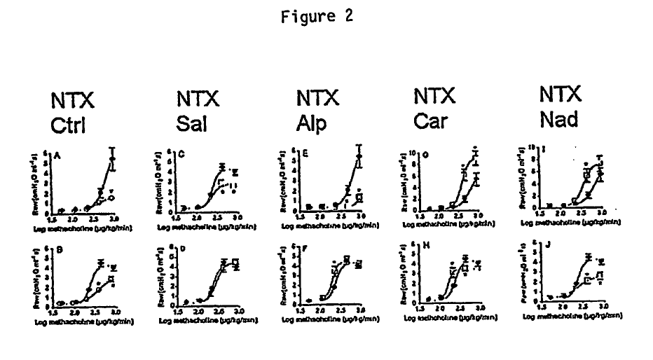

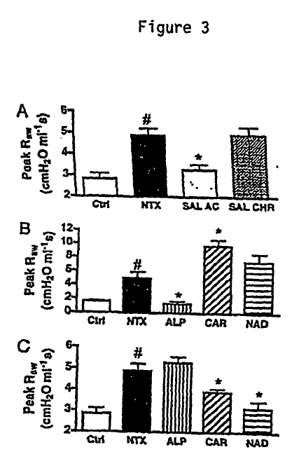

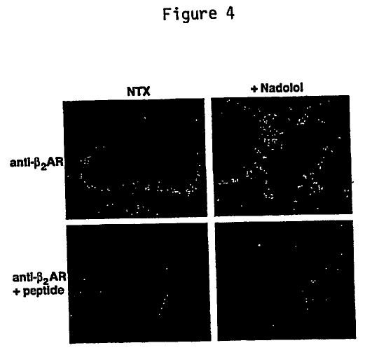

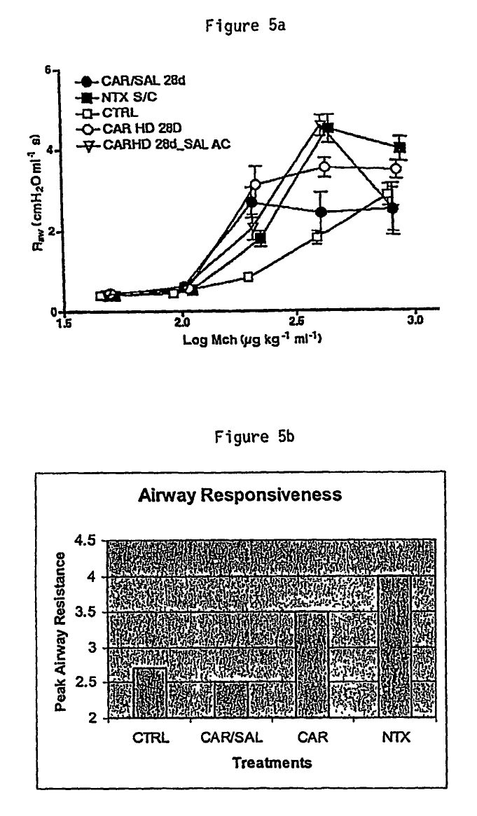

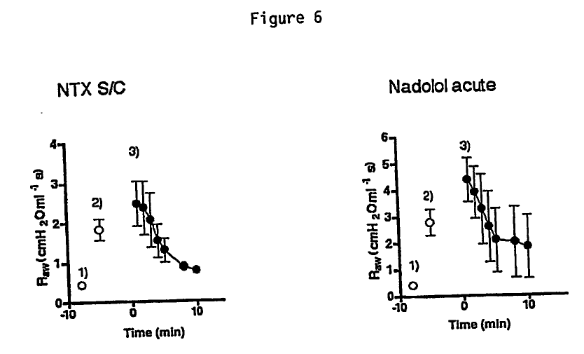

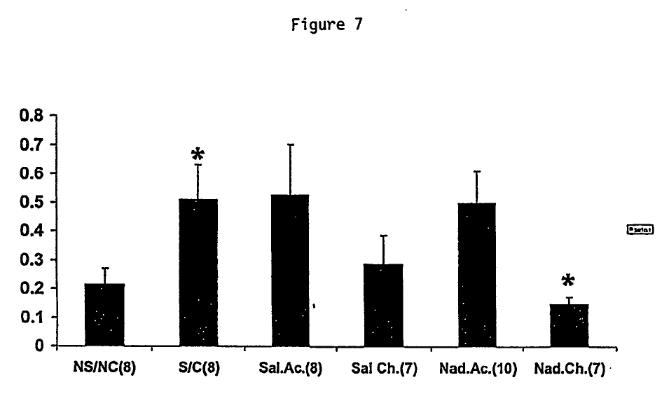

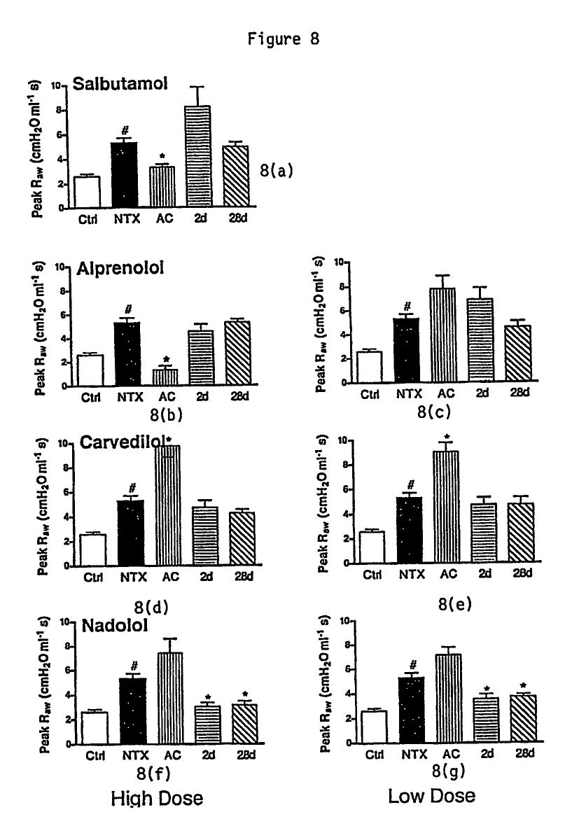

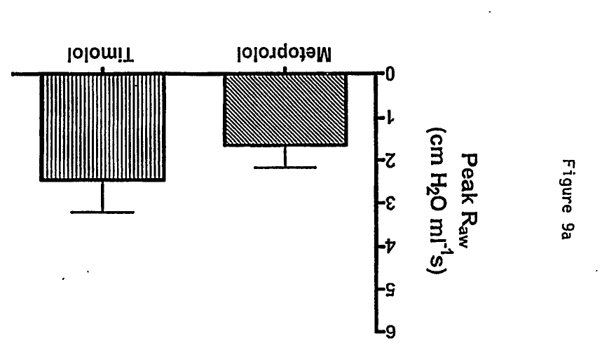

FIG. 2J is a similar graph showing that chronic administration of nadolol reduced the responsiveness of asthmatic mice to methacholine provocation, again, similar to that observed for carvedilol.

FIG. 3 is a graph showing the effects of administration of β-adrenergic receptor ligands on the peak airway responsiveness to cholinergic stimulation ((A), after treatments with the β-adrenergic agonist salbutamol; (B), after acute treatments with β-adrenergic receptor inverse agonists; and (C) after chronic treatment with β-adrenergic receptor inverse agonists.

FIG. 4 is an epifluorescent photomicrograph showing an increase in β-adrenergic receptor density upon treatment with nadolol.

FIG. 5A is a graph showing the effect of combination therapy with carvedilol and salbutamol on airway hyperresponsiveness in asthmatic mice challenged with methacholine.

FIG. 5B is a summary graph showing the results presented in FIG. 5A.

FIG. 6 is a graph showing the effect of acute combination therapy with nadolol and aminophylline on airway hyperresponsiveness in asthmatic mice challenged with methacholine.

FIG. 7 is a graph showing the ratio of phospholipase C to actin in mice treated with various treatments, including long-term nadolol administration, to show that long-term nadolol administration decreases the activity of phospholipase C.

FIG. 8A is a graph showing the effects of salbutamol upon airway hyperresponsiveness.

FIG. 8B is a graph showing the effects of high-dose alprenolol upon airway hyperresponsiveness.

FIG. 8C is a graph showing the effects of low-dose alprenolol upon airway hyperresponsiveness.

FIG. 8D is a graph showing the effects of high-dose carvedilol upon airway hyperresponsiveness.

FIG. 8E is a graph showing the effects of low-dose carvedilol upon airway hyperresponsiveness.

FIG. 8F is a graph showing the effects of high-dose nadolol upon airway hyperresponsiveness.

FIG. 8G is a graph showing the effects of low-dose nadolol upon airway hyperresponsiveness.

### DETAILED DESCRIPTION OF THE INVENTION

As used herein, in the generally accepted two-state model of receptor theory, the term “agonist” is defined as a substance that has an affinity for the active site of a receptor and thereby preferentially stabilizes the active state of the receptor, or a substance, including, but not limited to, drugs, hormones, or neurotransmitters, that produces activation of receptors and enhances signaling by those receptors. Irrespective of the mechanism or mechanisms of action, an agonist produces activation of receptors and enhances signaling by those receptors.

As used herein, in the two-state model of receptor theory, the term “antagonist” is defined as a substance that does not preferentially stabilize either form of the receptor, active, or inactive, or a substance, including, but not limited to, drugs, hormones, and neurotransmitters, that prevents or hinders the effects of agonists and/or inverse agonists. Irrespective of the mechanism or mechanisms of action, an antagonist prevents or hinders the effects of agonists and/or inverse agonists.

As used herein, in the two-state model of receptor theory, the term “inverse agonist” is defined as a substance that has an affinity for the inactive state of a receptor and thereby preferentially stabilizes the inactive state of the receptor, or a substance, including, but not limited to, drugs, hormones, or neurotransmitters, that produces inactivation of receptors and/or prevents or hinders activation by agonists, thereby reducing signaling from those receptors.

As used herein, the term “concurrent administration” refers to the administration of two or more active agents sufficiently close in time to achieve a combined therapeutic effect that is preferably greater than that which would be achieved by the administration of either agent alone. Such concurrent administration can be carried out simultaneously, e.g., by administering the active agents together in a common pharmaceutically acceptable carrier in one or more doses.

The term “subject,” as used herein, refers to human or animal species. In general, methods and compositions according to the present invention can be used to treat not only humans, but also socially or economically important animal species such as cows, horses, sheep, pigs, goats, dogs, and cats. Unless specified, methods and compositions according to the present invention are not limited to treatment of humans.

The term “therapeutically effective amount,” as used herein, refers to an amount of a therapeutic agent or composition effective to treat, ameliorate, or prevent a desired disease or condition, or to exhibit a detectable therapeutic or preventative effect. The effect can be detected by, for example, chemical markers, antigen levels, or changes in physiological indicators such as airway resistance. Therapeutic effects also include reduction in physical symptoms, such as decreased bronchoconstriction or decreased airway resistance, and can include subjective improvements in well-being noted by the subjects or their caregivers. The precise therapeutically effective amount for a subject will depend upon the subject's size, weight, and health, the nature and extent of the condition affecting the subject, and the therapeutics or combination of therapeutics selected for administration, as well as variables such as liver and kidney function that affect the pharmacokinetics of administered therapeutics. Thus, it is not useful to specify an exact effective amount in advance. However, the effective amount for a given situation can be determined by routine experimentation and is within the judgment of the clinician.

The present invention provides for a general strategy based on previous unrecognized pharmacology of the effects of inverse agonists on G protein-coupled receptors. Compounds producing an acutely detrimental effect via G protein-coupled receptors may provide a therapeutically beneficial effect with chronic administration and indicate that the chronic effect of the compounds cannot be predicted from their acute effects. Therefore, the present invention provides methods for treating diseases and conditions associated with the activity of G protein-coupled receptors. It further provides screening methods for detecting active agents that are inverse agonists and that are capable of treating diseases and conditions associated with the activity of G protein coupled receptors.

The basis of this strategy is the recognition of the existence of inverse agonists and the understanding of the effect that chronic treatment with inverse agonists has on receptor function. Receptors, such as β-ardenoceptors that respond to adrenalin (epinephrine), typically exist in an equilibrium between two states, an active state and an inactive state. When receptors bind to agonists, such as adrenalin for the β-ardenoceptors, they stop them from cycling back into the inactive state, thus shifting the equilibrium between the active and inactive states according to the Law of Mass Action. This occurs because those receptors bound to agonists are removed from the equilibrium. Typically, antagonists bind to the receptors, but prevent the binding of agonists. However, molecules known as “inverse agonists” bind to the receptors in the inactive state, causing the equilibrium between the active and the inactive states to shift toward the inactive state.

In classical receptor theory, two classes of G protein-coupled receptor (GPCR) ligands were considered: agonist and antagonist. Receptors were believed to exist in a single quiescent state that could only induce cellular signaling upon agonist binding to produce an activated receptor state. In this model, binding by antagonists produced no cellular signaling but simply prevented receptors from being bound and activated by agonists. Then, Costa and Herz demonstrated that receptors could be manipulated into a constitutive or spontaneously active state that produced cellular signaling in the absence of agonist occupation. They also provided evidence that certain compounds inactivate those spontaneously active receptors (T. Costa & A. Herz, “Antagonists with Negative Intrinsic Activity at δ Opioid Receptors Coupled to GTP-Binding Proteins,” Proc. Natl. Acad. Sci. USA 86: 7321-7325 (1989)). There is further evidence that GPCRs exist in constitutively or spontaneously active states that are inactivated to some degree by inverse agonists (R. A. de Ligt et al., “Inverse Agonism at G Protein-Coupled Receptors: (Patho)physiological Relevance and Implications for Drug Discovery,” Br. J. Pharmacol. 130: 1-12 (2000); G. Milligan et al., “Inverse Agonism: Pharmacological Curiosity or Potential Therapeutic Strategy?,” Trends Pharmacol. Sci. 16: 10-13 (2000)).

The basis of the strategy of this embodiment of the invention is the recognition of the existence of inverse agonists and the understanding of the effect that chronic treatment with inverse agonists has on receptor function. What is an inverse agonist and how does it function? Receptors, such as β-adrenergic receptors that respond to adrenalin (epinephrine), typically exist in an equilibrium between two states, an active state and an inactive state. When receptors bind to agonists, such as adrenalin for the β-ardenoceptors, they stop them from cycling back into the inactive state, thus shifting the equilibrium between the active and inactive states according to the Law of Mass Action. This occurs because those receptors bound to agonists are removed from the equilibrium. Typically, antagonists bind to the receptors, but prevent the binding of agonists. However, molecules known as “inverse agonists” bind to the receptors in the inactive state, causing the equilibrium between the active and the inactive state to shift toward the inactive state. This is not merely a matter of blocking agonist binding.

Moreover, there is a population of spontaneously active receptors in vivo. These receptors provide a baseline constitutive level of activity; the activity is never entirely “off.”

As indicated above, it has been well demonstrated that chronic administration of β-adrenergic agonists causes agonist-dependent desensitization. Upon acute administration of β-agonists, adrenergic receptors are internalized, thereby preventing them from being restimulated further for pulmonary relaxation. With chronic administration of β-agonists, there is actually a downregulation in the total number of β-adrenergic receptors. The consequence may be the observed loss of responsiveness seen in asthmatic patients on long-acting β-agonists, and referred to as tolerance or tachyphylaxis, as described above.

The treatment methods of the present invention are based on the discovery that a chronic administration of an inverse agonist has the effect of upregulating the population of active β-adrenergic receptors. The observed activity may be due to the receptor's constitutive baseline activity or the combined effect of increased level of receptors responding to endogenous agonists. This leads to the seemingly paradoxical result that the administration of a drug that would appear, at first blush, to degrade a physiological function, such as by causing airway hyperresponsiveness in asthma, can, if administered chronically, enhances that physiological function by upregulating the population of spontaneously active β-adrenergic receptors associated with that physiological function. This is a specific application of the principle of “paradoxical pharmacology.”

Among the diseases, syndromes, and conditions that Applicant believes are susceptible to improved means of treatment by the use of inverse agonists are pulmonary airway diseases, obesity, Alzheimer's disease (characterized by hypofunction of muscarinic receptors), and chronic pain, often treated by the use of opiate receptor agonists. All of these diseases, syndromes, and conditions are characterized by the development of tolerance or tachyphylaxis on continued agonist administration. The common thread of these conditions is a deficiency or hypofunction in GPCR signaling.

Other physiological processes, not necessarily pathological in nature, are also susceptible to manipulation by the administration of inverse agonists. For example, the sense of smell is also characterized by tolerance or reduction of signaling after chronic exposure to the same odor. This has often been considered psychological, and called accommodation, but may also have a basis in receptor biochemistry. Therefore, the administration of inverse agonists along with agonists (i.e., the odorific substance itself), can prolong the detection of the odor.

Along these lines, the use of cardioselective β inverse agonists (those with a preference for the β-adrenergic receptor subtype) has been demonstrated to be safe in hypertensive and congestive heart failure (CHF) patients with chronic airway obstructive pulmonary disease (COPD).

Multiple studies have demonstrated that chronic administration of cardioselective β inverse agonists does not change pulmonary function of CHF patients with COPD or asthma. Forced expiratory volume (FEV), a standard measure of pulmonary function, was essentially unchanged in patients treated with cardioselective β inverse agonists. These data indicate that chronic administration of cardioselective β inverse agonists is safe in CHF patients with pulmonary airway disease. However, these drugs are not preferred for reducing or altering the symptoms of pulmonary airway disease.

In U.S. Pat. No. 5,116,867 to Klein et al., incorporated herein by this reference, D-propranolol or racemic mixtures composed of 85% or more of the D form was proposed for the treatment of asthma. The D-form of propranolol was 1/100 as potent as the L-form in inhibiting the β-adrenergic receptor. In contrast, this patent specifies the use of the active form or of racemic mixtures containing 50% or more of the active β-ardenergic antagonist.

In U.S. Pat. No. 6,284,800 to Broder et al., incorporated herein by this reference, the D forms of propranolol, metoprolol, carvedilol, or bisoprolol were proposed for the treatment of asthma. Experiments were performed comparing the D-form versus the L-form of propranolol, demonstrating that acute administration of D-propranolol was beneficial in inhibiting antigen-induced bronchoconstriction and reducing airway hyperresponsiveness. In contrast, acute administration of the L-form increased specific lung resistance as expected for an active β-adrenergic agonist. The D form of propranolol was inactive with respect to β-adrenergic receptors. Consequently, U.S. Pat. No. 6,284,800 does not deal with inverse agonism.

PCT Patent Publication No. WO 02/29534, by Bond, had proposed compounds with β₁ and/or β₂ antagonist activity that inhibited β-adrenergic receptors to treat allergic and inflammatory disorders including asthma and chronic obstructive pulmonary disease. Experiments were performed in which asthmatic mice were chronically treated with compounds characterized as β-antagonists, including alprenolol, carvedilol, and ICI-118,551. Then, tracheas from the mice were excised and contraction of the tracheas in response to methacholine was monitored as a surrogate for an asthma attack. The most effective compound was alprenolol, followed by carvedilol, then ICI-118,551.

More physiologically relevant experiments in asthmatic mice performed by the inventor in the present application have demonstrated that alprenolol, originally thought to be beneficial chronically, does not reduce airway hyperresponsiveness compared to untreated asthmatic mice. Even though alprenolol is a β-adrenergic antagonist, it has partial agonist activity. Carvedilol is a β₁/β₂ non-selective adrenergic antagonist with α₁-adrenergic antagonist activity. In the new experiments reported in the present application, chronic administration of carvedilol does reduce airway hyperresponsiveness, which would be beneficial to asthmatics, but it also shifts the sensitivity of the responsiveness to methacholine to lower concentrations, which could be detrimental to asthmatics.

Moreover, in the experiments reported in PCT Patent Publication No. WO 02129534, tracheas were excised from mice, leaving behind the vast majority of the pulmonary airways. In mice, the trachea contains almost exclusively only β₁ adrenergic receptors whereas the remainder of the airways is a mixture of β₁ and β₂ adrenergic receptors. In contrast, human airways, both the trachea and the smaller airways, contain almost exclusively β₂ receptors. Consequently, the experiments reported in PCT Patent Publication No. WO 02/29534 have little predictive value for human asthma. The experiments reported in the present application more closely reflect human physiology.

β-adrenergic antagonist drugs or “beta blockers” are treated as having the same activity in conventional pharmacology. Beta blockers are further classified based on their selectivity or lack thereof for either the βhd 1 (termed “cardioselective”) or β₁/β₂ (“nonselective”) or β₂ selective only. Additionally, beta blockers can be classified as to whether or not they have partial agonist activity or are actually inverse agonists. The latter definition is based on the new appreciation, recited in the present application, that many G-coupled protein receptors, including the β-adrenergic receptors, exhibit low level spontaneous activity that can be further prevented by the binding of the inverse agonists to the receptor. This distinction was not made in PCT Patent Publication No. WO 02/29534, which referred simply to “antagonists.”

Despite this knowledge of the subclasses of beta blockers in the field, many scientists have continued to treat compounds from the different subclasses as one class. An example of this is the clinical testing in 1998-1999 of the beta blocker bucindolol for congestive heart failure. Previously, two other beta blockers, metoprolol and carvedilol, had been clinically tested and demonstrated significant mortality reduction in patients with CHF. Bucindolol failed to demonstrate any benefit over placebo, and thus clinical testing was discontinued. The inventor of the present application notes that both metoprolol and carvedilol are p-inverse agonists whereas bucindolol is a neutral antagonist with partial agonist activity. Consequently, the inventor of the present application would predict that only β-adrenergic inverse agonists would be effective in treatment of CHF. In the same vein, the inventor of the present application predicts that only β-adrenergic inverse agonists will be effective for chronic treatment of asthma airway hyperresponsiveness. This distinction is not made or suggested in PCT Patent Publication No. WO 02129534.

Beta antagonists were also once contraindicated for congestive heart failure (CHF). However, extensive clinical trials have repudiated this and now the beta antagonist carvedilol is approved by the FDA as a first-line therapy for CHF. Clinicians developed a very slow dosage ramping scheme to administer carvedilol safely to prevent any acute responses.

It is also well documented that chronic administration of beta adrenergic agonists cause agonist-dependent desensitization. Upon acute administration of beta agonists, adrenergic receptors are internalized thereby preventing them from being restimulated further for pulmonary relaxation. With chronic administration of beta agonists, there is actually a downregulation in the total number of beta adrenergic receptors. The consequence may be the observed loss of responsiveness seen in asthmatic patients on long-acting beta agonists.

The treatment methods of the present invention are based on the discovery that chronic administration of an inverse agonist has the effect of upregulating the population of spontaneously active GPCRs. This leads to the paradoxical result that the administration of a drug that would appear, at first blush, to degrade a physiological function, such as by causing airway hyperresponsiveness in asthma, can, if administered chronically, enhance that physiological function by upregulating the population of spontaneously active GPCRs associated with that physiological function. This is a specific example of the application of the principle of “paradoxical pharmacology.”

Accordingly, in general, one aspect of the present invention is a method for treating a disease or condition associated with the activity of a G protein coupled receptor (GPCR) comprising administering an inverse agonist for the GPCR to an organism with a disease or condition associated with the activity of the GPCR in a quantity and for a period that causes an increase in the population of GPCRs, either spontaneously active or those that are available and activated by an endogenous agonist or by an exogenous agonist, associated with that physiological function, thereby producing a therapeutic effect to ameliorate the disease or condition. The particular exogenous agonist to be used depends on the receptor and the disease or condition to be treated. Another aspect of the invention is administering an inverse agonist for the GPCR to an organism with a disease or condition associated with the activity of the GPCR in a quantity and for a period that prevents the decrease in the population of GPCRs due to the presence of either exogenous or endogenous agonist.

Typically, the chronic administration of an inverse agonist provides a therapeutic benefit equivalent or greater than the therapeutic effect of the administration of an acute agonist.

Typically, the method of administration of the inverse agonist results in continuous levels of the inverse agonist in the bloodstream of the organism to which the inverse agonist is being administered.

The disease or condition associated with the activity of the GPCR can be a pulmonary airway disease. Typically, the pulmonary airway disease is asthma. Alternatively, the pulmonary airway disease is allergic rhinitis, bronchiectasis, bronchitis, chronic obstructive pulmonary disease (COPD), Churg-Strauss syndrome, pulmonary sequelae of cystic fibrosis, emphysema, or pneumonia.

When the disease or condition associated with the activity of a GPCR is a pulmonary airway disease, the therapeutic effect can be a reduction in pulmonary airway constriction hyperresponsiveness.

When the disease or condition associated with the activity of a GPCR is a pulmonary airway disease, the GPCRs can be β₂-adrenergic receptors. The therapeutic effect can be an upregulation of the population of pulmonary β₂-adrenergic receptors. The therapeutic effect can also be increased pulmonary airway relaxation responsiveness to β₂-adrenergic agonist drugs.

Especially preferred for use according to the invention are the inverse β-adrenergic agonists: nadolol, e.g., as the hydrochloride: bupranolol, e.g., as the hydrochloride; butoxamine, e.g., as the hydrochloride; carazolol; e.g., as the hydrochloride; carvedilol; , e.g., as the hydrochloride; ICI-118,551, i.e., as the hydrochloride; levobunolol, e.g., as the hydrochloride; metoprolol, e.g., as the succinate or tartrate; propranolol, e.g., as the hydrochloride; sotalol, e.g., as the hydrochloride; timolol, e.g., as the hydrochloride; CGβ-20712A; and the salts, solvates, analogues, congeners, bioisosteres, hydrolysis products, metabolites, precursors, and prodrugs thereof. Suitable inverse agonists for use in methods and compositions according to the present invention that are inverse β-adrenergic agonists can be either selective β₁-adrenergic inverse agonists, selective β₂-adrenergic inverse agonists, or non-selective inverse agonists that have inverse agonist activity at both β₁-adrenergic and β₂-adrenergic receptors. In some applications, however, selective β₂-adrenergic inverse agonists are preferred. Particularly preferred inverse agonists are carvedilol and nadolol. A most particularly preferred inverse agonist is nadolol. As used herein, the recitation of an inverse agonist compound, or, where appropriate, an agonist compound, includes all pharmaceutically acceptable salts of that inverse agonist compound or agonist compound unless excluded. Thus, the recitation of nadolol as the hydrochloride does not exclude other pharmaceutically acceptable salts that have been prepared or that can be prepared.

The inverse agonists can be in pure or substantially pure enantiomeric or diastereomeric form or can be racemic mixtures. In many cases, the active form of such compounds is the L form when there is only one chiral center. In the case of nadolol, which has three chiral centers and potentially 12 isomers, though, typically, only two are formed during synthesis, the most active form is the RSR form of nadolol. The recitation of “nadolol” herein includes all active isomers, including diasteromers of nadolol, unless the contrary is stated.

Specifically, also expected to be within the scope of the invention are analogues of nadolol of formula (I) wherein R₁ is hydrogen or lower alkyl, R₂ is hydrogen or lower alkyl, and m and n are 1 to 3, with the proviso that where R₁ and R₂ are both hydrogen and m is 1, n is other than 1. As used herein, the term “lower alkyl” is defined as a straight or branched hydrocarbyl residue of 1-6 carbon atoms.

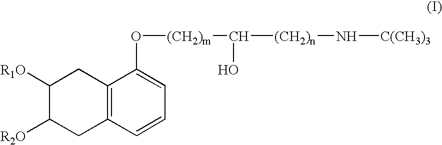

Also specifically expected to be within the scope of the invention are analogues of carvedilol of formula (II) wherein R₁ is hydrogen or lower alkyl, R₂ is hydrogen or lower alkyl, and R₃ is hydrogen or lower alkyl, with the proviso that all of R₁, R₂, and R₃ are not all hydrogen.

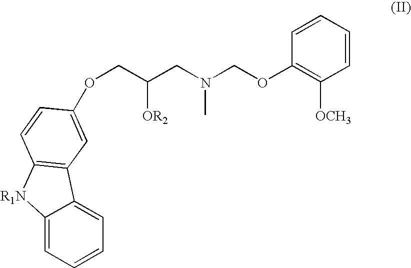

Also expected to be within the scope of the invention are analogues of timolol of formula (III) wherein R₁ is hydrogen or lower alkyl and R₂ is hydrogen or lower alkyl, with the proviso that both R₁ and R₂ are not hydrogen.

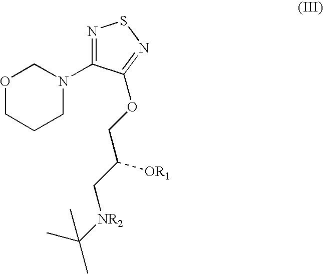

Further expected to be within the scope of the invention are analogues of metoprolol of formula (IV) wherein R₁ is hydrogen or lower alkyl and R₂ is hydrogen or lower alkyl, with the proviso that both R₁ and R₂ are not hydrogen.

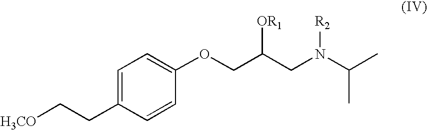

In the case of salts, it is well known that organic compounds, including compounds having activities suitable for methods according to the present invention, have multiple groups that can accept or donate protons, depending upon the pH of the solution in which they are present. These groups include carboxyl groups, hydroxyl groups, amino groups, sulfonic acid groups, and other groups known to be involved in acid-base reactions. The recitation of a compound or analogue includes such salt forms as occur at physiological pH or at the pH of a pharmaceutical composition unless specifically excluded.

Similarly, prodrug esters can be formed by reaction of either a carboxyl or a hydroxyl group on compounds or analogues suitable for methods according to the present invention with either an acid or an alcohol to form an ester. Typically, the acid or alcohol includes a lower alkyl group such as methyl, ethyl, propyl, isopropyl, butyl, isobutyl, and tertiary butyl. These groups can be substituted with substituents such as hydroxy, or other substituents. Such prodrugs are well known in the art and need not be described further here. The prodrug is converted into the active compound by hydrolysis of the ester linkage, typically by intracellular enzymes. Other suitable groups that can be used to form prodrug esters are well known in the art. For example prodrugs can include amides prepared by reaction of the parent acid compound with a suitable amine. In some cases it is desirable to prepare double ester type prodrugs such as (acyloxy) alkyl esters or ((alkoxycarbonyl)oxy)alkyl esters. Suitable esters as prodrugs include, but are not necessarily limited to,methyl, ethyl, propyl, isopropyl, n-butyl, isobutyl, tert-butyl, morpholinoethyl, and N,N-diethylglycolamido. Methyl ester prodrugs may be prepared by reaction of the acid form of a compound having a suitable carboxylic acid group in a medium such as methanol with an acid or base esterification catalyst (e.g., NaOH, H₂ SO₄). Ethyl ester prodrugs are prepared in similar fashion using ethanol in place of methanol. Morpholinylethyl ester prodrugs may be prepared by reaction of the sodium salt of a suitable compound (in a medium such as dimethylformamide) with 4-(2-chloroethyl)morphine hydrochloride (available from Aldrich Chemical Co., Milwaukee, Wis. USA.

Pharmaceutically acceptable salts include acid salts such as hydrochloride, hydrobromide, hydroiodide, sulfate, phosphate, fumarate, maleate, acetates, citrates, lactates, tartrates, sulfamates, malonate, succinate, tartrate, methanesulfonates, ethanesulfonates, benzenesulfonates, p-toluenesulfonates, cyclohexylsulfamates, quinates, formates, cinnamates, picrates, and other suitable salts. Such salts can be derived using acids such as hydrochloric acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, and quinic acid.

Pharmaceutically acceptable salts also include salts with bases such as alkali metal salts such as sodium or potassium, as well as pyridine salts, ammonium salts, piperazine salts, diethylamine salts, nicotinamide salts, calcium salts, magnesium salts, zinc salts, lithium salts, methylamino salts, triethylamino salts, dimethylamino salts, and tris(hydroxymethyl) aminomethane salts.

In addition, where compounds recited above are optically active, both the optically active form and the racemic mixture are encompassed by the present invention unless the racemic mixture is specifically excluded.

Typically, the method of administration of the β₂-adrenergic inverse agonist results in continuous levels of the β₂-adrenergic inverse agonist in the bloodstream of the subject. Typically, the method exerts a therapeutic effect that is an upregulation of pulmonary β₂-adrenergic receptors. Typically, the method exerts a therapeutic effect that is increased pulmonary airway relaxation responsiveness to β₂-adrenergic agonist drugs. This provides for combination therapy, discussed in detail below.

The β-adrenergic inverse agonist can be administered in conjunction with one or more pharmaceutical excipients. The pharmaceutical excipients can include, but are not necessarily limited to, calcium carbonate, calcium phosphate, various sugars or types of starch, cellulose derivatives, gelatin, vegetable oils, polyethylene glycols and physiologically compatible solvents. Other pharmaceutical excipients are well known in the art. The β-adrenergic inverse agonist can be administered in conjunction with one or more pharmaceutically acceptable carriers. Exemplary pharmaceutically acceptable carriers include, but are not limited to, any and/or all of solvents, including aqueous and non-aqueous solvents, dispersion media, coatings, antibacterial and/or antifungal agents, isotonic and/or absorption delaying agent, and/or the like. The use of such media and/or agents for pharmaceutically active substances is well known in the art. Except insofar as any conventional medium, carrier, or agent is incompatible with the active ingredient or ingredients, its use in a composition according to the present invention is contemplated. Supplementary active ingredients can also be incorporated into the compositions, especially as described below under combination therapy. For administration of any of the compounds used in the present invention, preparations should meet sterility, pyrogenicity, general safety, and purity standards as required by the FDA Office of Biologics Standards or by other regulatory organizations regulating drugs.

Thus, the β-adrenergic inverse agonist can be formulated for oral, sustained-release oral, buccal, sublingual, inhalation, insufflation, or parenteral administration.

If the β-adrenergic inverse agonist is administered orally, either in a conventional or a sustained-release preparation, it is typically administered in a conventional unit dosage form such as a tablet, a capsule, a pill, a troche, a wafer, a powder, or a liquid such as a solution, a suspension, a tincture, or a syrup. Oral formulations typically include such normally employed excipients as, for example, pharmaceutical grades of mannitol, lactose, starch, magnesium stearate, sodium saccharin, cellulose, magnesium carbonate, and other conventional pharmaceutical excipients. In certain defined embodiments, oral pharmaceutical compositions will comprise an inert diluent and/or assimilable edible carrier, and/or they may be enclosed in hard or soft shell gelatin capsules. Alternatively, they may be compressed into tablets. As another alterative, particularly for veterinary practice, they can be incorporated directly into food. For oral therapeutic administration, they can be incorporated with excipients or used in the form of ingestible tablets, buccal tablets, dragees, pills, troches, capsules, wafers, or other conventional dosage forms.

The tablets, pills, troches, capsules, wafers, or other conventional dosage forms can also contain the following: a binder, such as gum tragacanth, acacia, cornstarch, sorbitol, mucilage of starch, polyvinylpyrrolidone, or gelatin; excipients or fillers such as dicalcium phosphate, lactose, microcrystalline cellulose, or sugar; a disintegrating agent such as potato starch, croscarmellose sodium, or sodium starch glycolate, or alginic acid; a lubricant such as magnesium stearate, stearic acid, talc, polyethylene glycol, or silica; a sweetening agent, such as sucrose, lactose, or saccharin; a wetting agent such as sodium lauryl sulfate; or a flavoring agent, such as peppermint, oil of wintergreen, orange flavoring, or cherry flavoring. When the dosage unit form is a capsule, it can contain, in addition to materials of the above types, a liquid carrier. Various other materials can be present as coatings or to otherwise modify the physical form and properties of the dosage unit. For instance, tablets, pills, or capsules can be coated with shellac, sugar, or both. The pharmaceutical compositions of the present invention may be manufactured in a manner that is itself known, e.g., by means of conventional mixing, dissolving, granulating, dragee-making, levitating, emulsifying, encapsulating, entrapping or lyophilizing processes.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

In one alterative, a sustained-release formulation is used. Sustained-release formulations are well-known in the art. For example, they can include the use of polysaccharides such as xanthan gum and locust bean gum in conjunction with carriers such as dimethylsiloxane, silicic acid, a mixture of mannans and galactans, xanthans, and micronized seaweed, as recited in U.S. Pat. No. 6,039,980 to Baichwal, incorporated herein by this reference. Other sustained-release formulations incorporate a biodegradable polymer, such as the lactic acid-glycolic acid polymer recited in U.S. Pat. No. 6,740,634 to Saikawa et al., incorporated herein by this reference. Still other sustained-release formulations incorporate an expandable lattice that includes a polymer based on polyvinyl alcohol and polyethylene glycol, as recited in U.S. Pat. No. 4,428,926 to Keith, incorporated herein by this reference. Still other sustained-release formulations are based on the Eudragit™ polymers of Rohm & Haas, that include copolymers of acrylate and methacrylates with quaternary ammonium groups as functional groups as well as ethylacrylate methylmethacrylate copolymers with a neutral ester group. A particularly-preferred extended release composition suitable for use in methods according to the present invention is an extended-release composition that contains nadolol as its active ingredient.

Oral liquid preparations can be in the form of, for example, aqueous or oily suspensions, solutions, emulsions, syrups, tinctures, or elixirs, or can be presented as a dry product for reconstitution with water or other suitable vehicles before use. Such liquid preparations can contain conventional additives such as suspending agents, for example, sorbitol syrup, methylcellulose, glucose/sugar syrup, gelatin, hydroxymethylcellulose, carboxymethylcellulose, aluminum stearate gel, or hydrogenated edible fats; emulsifying agents, such as lecithin, sorbitan monooleate, or acacia; non-aqueous vehicles (which may include edible oils), for example, almond oil, fractionated coconut oil, oily esters, propylene glycol, or ethyl alcohol; or preservatives, for example, methylparaben, propylparaben, or sorbic acid. The preparations can also contain buffer salts, flavoring, coloring, or sweetening agents (e.g., mannitol) as appropriate.

One skilled in the art recognizes that the route of administration is an important determinant of the rate of efficiency of absorption. For example, the alimentary route, e.g., oral, rectal, sublingual, or buccal, is generally considered the safest route of administration. The delivery of the drugs into the circulation is slow, thus eliminating rapid high blood levels of the drugs that could potentially have adverse acute effects. Although this is considered the safest route of administration, there are several disadvantages. One important disadvantage is that the rate of absorption varies, which is a significant problem if a small range in blood levels separates a drug's desired therapeutic effect from its toxic effect, i.e., if the drug has a relatively low therapeutic index. Also, patient compliance is not always ensured, especially if the rectal route of administration is chosen or if oral administration is perceived by the patient as unpleasant. Furthermore, with oral administration, extensive hepatic metabolism can occur before the drug reaches its target site. Another route of administration is parenteral, which bypasses the alimentary tract. One important advantage of parenteral administration is that the time for the drug to reach its target site is decreased, resulting in a rapid response, which is essential in an emergency. Furthermore, parenteral administration allows for delivery of a more accurate dose. Parenteral administration also allows for more rapid absorption of the drug, which can result in increased adverse effects. Unlike alimentary administration, parenteral administration requires a sterile formulation of the drug and aseptic techniques are essential. The most significant disadvantage to parenteral administration is that it is not suitable for insoluble substances. In addition to alimentary and parenteral administration routes, topical and inhalation administrations can be useful. Topical administration of a drug is useful for treatment of local conditions; however, there is usually little systemic absorption. Inhalation of a drug provides rapid access to the circulation and is the common route of administration for gaseous and volatile drugs, or drugs that can be vaporized or nebulized. It is also a desired route of administration when the targets for the drug are present in the pulmonary system.

When compounds are formulated for parenteral administration, e.g., formulated for injection via the intravenous, intramuscular, subcutaneous, intralesional, or intraperitoneal routes, many options are possible. The preparation of an aqueous composition that contains an effective amount of the β-adrenergic inverse agonist as an active ingredient will be known to those of skill in the art. Typically, such compositions can be prepared as injectables, either as liquid solutions and/or suspensions. Solid forms suitable for use to prepare solutions and/or suspensions upon the addition of a liquid prior to injection can also be prepared. The preparations can also be emulsified.

The pharmaceutical forms suitable for injectable use include sterile aqueous solutions and/or dispersions; formulations including sesame oil, peanut oil, synthetic fatty acid esters such as ethyl oleate, triglycerides, and/or aqueous propylene glycol; and/or sterile powders for the extemporaneous preparation of sterile injectable solutions and/or dispersions. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. In all cases the form must be sterile and/or must be fluid to the extent that the solution will pass readily through a syringe and needle of suitable diameter for administration. It must be stable under the conditions of manufacture and storage and must be preserved against the contaminating action of microorganisms, such as bacteria or fungi.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and/or mixtures thereof and/or in oils. Under ordinary conditions of storage and use, these preparations contain a preservative to prevent the growth of microorganisms. Suitable non-sensitizing and non-allergenic preservatives are well known in the art.

The carrier can also be a solvent and/or dispersion medium containing, for example, water, ethanol, a polyol (for example, glycerol, propylene glycol, and/or liquid polyethylene glycol, and/or the like), suitable mixtures thereof, and/or vegetable oils. The proper fluidity can be maintained for example, by the use of a coating, such as lecithin, by the maintenance of a suitable particle size in the case of a dispersion, and/or by the use of surfactants. The prevention of the action of microorganisms can be brought about by the inclusion of various antibacterial and/or antifungal agents, for example, parabens, chlorobutanol, phenol, sorbic acid, or thimerosal. In many cases it will be preferable to include isotonic agents, for example, sugars or sodium chloride. In many cases, it is preferable to prepare the solution in physiologically compatible buffers such as Hanks's solution, Ringer's solution, or physiological saline buffer.Prolonged absorption of the injectable compositions can be brought about by the use in the compositions of agents delaying absorption, for example, aluminum monostearate and/or gelatin.

Sterile injectable solutions are prepared by incorporating the active compounds in the required amount in the appropriate solvent with various of the other ingredients enumerated above, as required, followed by sterilization. Sterilization is typically performed by filtration. Generally, dispersions are prepared by incorporating the various sterilized active ingredients into a sterile vehicle which contains the basic dispersion medium and/or the other required ingredients. In the case of sterile powders for the preparation of sterile injectable solutions, the preferred methods of preparation are vacuum-drying and/or freeze-drying techniques that yield a powder of the active ingredients plus any additional desires ingredients from a previously sterile-filtered solution thereof. The preparation of more-concentrated or highly-concentration solutions for direct injection is also contemplated, where the use of dimethyl sulfoxide (DMSO) as solvent is envisioned to result in extremely rapid penetration, delivering high concentrations of the active agents to a small area if desired.

For parenteral administration in an aqueous solution, for example, the solution should be suitably buffered if necessary and/or the liquid diluent first rendered isotonic with sufficient saline, glucose, or other tonicity agent. These particular aqueous solutions are especially suitable for intravenous, intramuscular, subcutaneous, or intraperitoneal administration. In this connection, sterile aqueous media which can be employed will be known to those of skill in the art in light of the present disclosure. For example, one dosage could be dissolved in 1 mL of isotonic NaCl solution and either added to 1000 mL of hypodermoclysis fluid or injected into the proposed site of infusion (see, e.g., “Remington's Pharmaceutical Sciences” (15th ed.), pp. 1035-1038, 1570-1580). Some variation in dosage will necessarily occur depending on the condition of the subject being treated. The person responsible for administration will, in any event, determine the appropriate dose for the individual subject. Compounds and compositions according to the invention can also be formulated for parenteral administration by bolus injection or continuous infusion and can be presented in unit dose form, for instance as ampules, vials, small volume infusions, or pre-filled syringes, or in multi-dose containers with an added preservative.

Another route of administration of compositions according to the present invention is nasally, using dosage forms such as nasal solutions, nasal sprays, aerosols, or inhalants. Nasal solutions are usually aqueous solutions designed to be administered to the nasal passages in drops or sprays. Nasal solutions are typically prepared so that they are similar in many respects to nasal secretions, so that normal ciliary action is maintained. Thus, the aqueous nasal solutions usually are isotonic and/or slightly buffered in order to maintain a pH of from about 5.5 to about 6.5. In addition, antimicrobial preservatives, similar to those used in ophthalmic preparations, and/or appropriate drug stabilizers, if required, can be included in the formulation. Various commercial nasal preparations are known and can include, for example, antibiotics or antihistamines. Spray compositions can be formulated, for example, as aqueous solutions or suspensions or as aerosols delivered from pressurized packs, with the use of a suitable propellant, such as dichlorodifluoromethane, trichlorofluoromethane, dichlorotetrafluoroethane, 1,1,1,2,3,3,3-heptafluoropropane, 1,1,1,2-tetrafluoroethane, carbon dioxide, or other suitable gas.

Additional formulations that are suitable for other modes of administration include vaginal suppositories and/or pessaries. A rectal pessary or suppository can also be used. Suppositories are solid dosage forms of various weights or shapes, usually medicated, for insertion into the rectum, vagina, or urethra. After insertion, suppositories soften, melt, and/or dissolve into the cavity fluids. In general, for suppositories, traditional binders or carriers can include polyalkylene glycols, cocoa butter, or triglycerides.

Other dosage forms, including but not limited to ointments, creams, lotions, powders, or creams, can alternatively be used. Ointments and creams can, for example, be formulated with an aqueous or oily base with the addition of suitable gelling agents and/or solvents. Such bases, can thus, for example, include water and/or an oil such as liquid paraffin or a vegetable oil such as arachis (peanut) oil or castor oil or a solvent such as a polyethylene glycol. Thickening agents which can be used include soft paraffin, aluminum stearate, cetostearyl alcohol, polyethylene glycols, microcrystalline wax, and beeswax. Lotions can be formulated with an aqueous or oily base and will in general also contain one or emulsifying agents, stabilizing agents, dispersing agents, suspending agents, or thickening agents.

Powders for external application can be formed with the aid of any suitable powder base, for example, talc, lactose, or starch.

The same principles applying to pharmaceutical compositions, dosage forms, pharmaceutically acceptable carriers, fillers, excipients, routes of administration, and methods of administration, described above particularly with respect to β-adrenergic inverse agonists, are also applicable to the administration of inverse agonists to other GPCRs, as described below.

Because of the nature of the interaction between inverse agonists and the receptors with which they interact, the therapeutic response develops gradually over time as the receptor density in the affected tissues increases in response to the administration of inverse agonists. Therefore, in one particularly preferred alternative, the dosage is titrated at the start of administration with gradual increases. In other words, the inverse agonist is administered over time in a series of graduated doses starting with the lowest dose and increasing to the highest dose. When the highest dose is reached, the inverse agonist, such as a β-adrenergic inverse agonist, continues to be administered at that dose (the maintenance dose). For example, with nadolol administered orally, treatment can begin with 1 mg dosages, then progress through 3 mg, 5 mg, 10 mg, 15 mg, and then to higher maintenance dosages such as 25 mg, 30 mg, 50 mg, 70 mg, or 100 mg, depending on the particular condition to be treated, the severity, and the response of the condition to the treatment. Analogous dosing regimens can be used with other inverse agonists, the exact starting dose typically depending on the affinity of the inverse agonist for the binding site of the β-adrenergic receptor or other receptor for which the inverse agonist has affinity.

Accordingly, another aspect of the invention is a blister pack that includes a range of dosages from the lowest initial dose to the highest maintenance dose of an adrenergic inverse agonist for a GPCR, as described above. In general, such a blister pack comprises:

(1) a lower substrate;

(2) an intermediate dosage holder that is shaped to generate a plurality of cavities and that is placed over the lower substrate, the cavities being shaped to hold dosage forms of the inverse agonist;

(3) an upper substrate placed over the intermediate dosage holder that has a plurality of apertures, each aperture being located to accommodate a corresponding cavity; wherein the dosage forms are of graduated dosages starting with a lowest dose and proceeding to a highest dose; and

(4) dosage forms of the inverse agonist placed in the cavities.

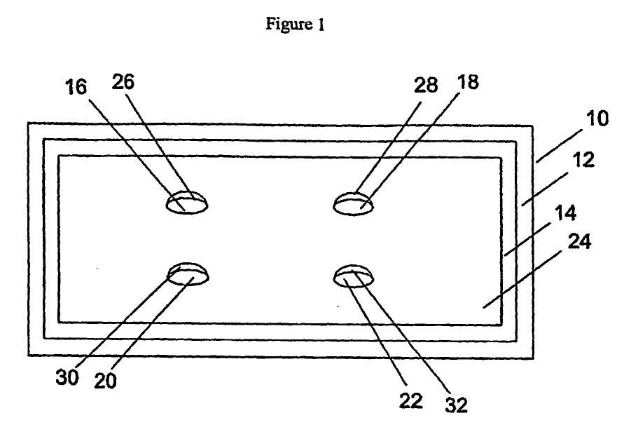

A suitable blister pack 10 is shown in FIG. 1 and includes a lower substrate 12 that is typically foil, an intermediate dosage holder 14 that is shaped to generate a plurality of cavities 16, 18, 20, and 22 shaped to hold the pills, capsules, or other dosage forms that is placed over the lower substrate, and an upper substrate 24 placed over the intermediate dosage holder 14 that has apertures 26, 28, 30, and 32, each aperture being located to accommodate the cavities 16, 18, 20, and 22. Only four cavities and apertures are shown here, but blister packs 10 according to the present invention can hold a larger number of dosage forms, such as 10, 20, or 30. Typically, either the lower substrate 12, the upper substrate 24, or both have printed instructions on it to identify the dosage of each pill, capsule, or other dosage forms, and to provide guidance to the patient as to the sequence to be followed in taking the pills, capsules, or other dosage forms. The intermediate dosage holder 14 is typically made of a transparent plastic or other transparent material so that the dosage forms can be viewed. The dosage forms can be of graduated doses, starting with a lowest dose and proceeding to a highest dose, which is generally the maintenance dose, as described above. Alternatively, the dosage forms can be of at least two dosages: (1) a maintenance dose that is the highest in a series of graduated doses; and (2) at least one backup restoration dose (to be used, e.g., if a dose is missed) or a lower dose to be taken in a specified condition. When the inverse agonist is a β-adrenergic inverse agonist, the specified condition can be, for example, the administration of an antibiotic, such as erythromycin or neomycin, where lower dosages are generally required.

Various factors must be taken into account in setting suitable dosages for β-adrenergic inverse agonists and for inverse agonists for other GPCRs. These factors include whether the patient is taking other medications that can alter the pharmacokinetics of the inverse agonists, either causing them to be degraded more rapidly or more slowly. In particular, when the inverse agonist is a β-adrenergic inverse agonist and if the patient is taking the antibiotics erythromycin or neomycin, it is typically necessary to decrease the maintenance dose. Another aspect of the invention is therefore a blister pack that has backup restoration doses and lower doses for use when the patient is taking these antibiotics.

Toxicity and therapeutic efficacy of inverse agonists, including β-adrenergic inverse agonists and inverse agonists for other GPCRs, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. The data obtained from these cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized.

For any compound used in the method of the invention, the therapeutically effective dose can be estimated initially from cell culture assays. For example, a dose can be formulated in animal models to achieve a circulating plasma concentration range that includes the IC₅₀ as determined in cell culture (i.e., the concentration of the test compound which achieves a half-maximal improvement in receptor signaling when chronic effects are considered). Such information can be used to more accurately determine useful doses in humans. Levels in plasma may be measured, for example, by HPLC.

The exact formulation, route of administration and dosage can be chosen by the individual physician in view of the patient's condition. (See e.g. Fingl et al., in The Pharmacological Basis of Therapeutics, 1975, Ch. 1 p. 1). It should be noted that the attending physician would know how to and when to terminate, interrupt, or adjust administration due to toxicity, or to organ dysfunctions. Conversely, the attending physician would also know to adjust treatment to higher levels if the clinical response were not adequate (precluding toxicity). The magnitude of an administered dose in the management of the disorder of interest will vary with the severity of the condition to be treated and to the route of administration. The severity of the condition may, for example, be evaluated, in part, by standard prognostic evaluation methods. Further, the dose and perhaps the dose frequency, will also vary according to the age, body weight, and response of the individual patient. A program comparable to that discussed above may be used in veterinary medicine.

Depending on the specific conditions being treated, such agents may be formulated and administered systemically or locally. Typically, administration is systemic. Techniques for formulation and administration may be found in Remington's Pharmaceutical Sciences, 18th ed., Mack Publishing Co., Easton, Pa. (1990). Suitable routes may include oral, rectal, transdermal, vaginal, transmucosal, or intestinal administration; administration by inhalation; parenteral delivery, including intramuscular, subcutaneous, intramedullary injections, as well as intrathecal, direct intraventricular, intravenous, intraperitoneal, intranasal, or intraocular injections, just to name a few. Typically, oral administration or administration by inhalation is preferred.

For injection, the agents of the invention may be formulated in aqueous solutions, For such transmucosal administration, penetrants appropriate to the barrier to be permeated are used in the formulation. Such penetrants are generally known in the art.

Use of pharmaceutically acceptable carriers to formulate the compounds herein disclosed for the practice of the invention into dosages suitable for systemic administration is within the scope of the invention. With proper choice of carrier and suitable manufacturing practice, the compositions of the present invention, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the invention to be formulated as tablets, pills, capsules, liquids, gels, syrups, slurries, suspensions and the like, for oral ingestion by a patient to be treated.

Pharmaceutical compositions suitable for use in the present invention include compositions wherein the active ingredients are contained in an effective amount to achieve its intended purpose. Determination of the effective amounts is well within the capability of those skilled in the art, especially in light of the detailed disclosure provided herein. In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions. The pharmaceutical compositions of the present invention may be manufactured in a manner that is itself known, e.g., by means of conventional mixing, dissolving, granulating, dragee-making, levitating, emulsifying, encapsulating, entrapping or lyophilizing processes.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Typically, in methods according to the present invention, the inverse agonist is administered in a daily dose or multiple times per day, depending on the half-life of the inverse agonist. Alternatively, the inverse agonist can be administered less frequently, such as every other day, every third day, every fourth day, every week, and the like. One skilled in the art of pharmacokinetics will recognize the importance of understanding the bioavailability and the half-life of a drug in relation to dosing of the particular drug. It is well known that a drug accumulates in the body if the time interval between doses is less than four of its half-lives, in which case, the total body stores of the drug are increased exponentially to a plateau or steady-state concentration. The average total body store of a drug at the plateau is a function of the dose, the interval between doses, the bioavailability of the drug, and the rate of the elimination of the drug. Thus, one of ordinary skill in the art is capable of determining the dose and interval of the dose for a given drug to achieve the desired effect.

The method for treating the disease or condition can further comprise the administration of an additional agent. In one alternative, the additional agent is a β₂-selective adrenergic agonist drug. The β₂-selective adrenergic agonist drug can be selected from the group consisting of albuterol, bitolterol, clenbuterol, clorprenaline, dobutamine, fenoterol, formoterol, isoetharine, isoprenaline, levabuterol, mabuterol, metaproterenol, pirbuterol, ritodrine, salbutamol, salmeterol, and terbutaline as well as the salts, solvates, analogues, congeners, bioisosteres, hydrolysis products, metabolites, precursors, and prodrugs thereof. The principle of combination therapy is supported by the data that shows that treatment with inverse agonists causes upregulation of the receptor number. In that case, co-treatment with an agonist would be expected to increase cellular signaling and restore normal function in those circumstances in which the pathological response is characterized by a deficiency in signaling. Along these lines, the inhibitory response of inverse agonists on airway resistance would be increased in magnitude by the co-administration of agonists. The potency of these agonists may be reduced due to the presence of the inverse agonist, but the overall magnitude of the response would be increased. This would prevent the desensitization often associated with chronic agonist treatment. The concentration of the agonist used can be determined from the effect of the concentration on receptor signaling.

This same rationale can be extended to other GPCR receptors, including, but not limited to, acetylcholine receptors, including muscarinic receptors, β₃-adrenergic receptors, and other receptors described above.

When combination therapy is used, the dosages of each member of the combination can be determined according to the principles described above. However, in many cases, fixed dose combinations are desirable and can be used. In the fixed dose combinations that apply the dosage of the inverse agonist can be determined as described above, while the desirable dosage of the appropriate agonist can be determined from the effect of particular concentrations of the agonist on receptor signaling.

In another alternative, the additional agent is a steroid. The steroid can be beclomethasone, budesonide, ciclesonide, flunisolide, fluticasone, methylpredisolone, prednisolone, prednisone, or triamcinolone, as well as the salts, solvates, analogues, congeners, bioisosteres, hydrolysis products, metabolites, precursors, and prodrugs thereof.

In yet another alternative, the additional agent is an anticholinergic drug. The anticholinergics especially preferred for use according to the invention include, but are not necessarily limited to, muscarinic receptor antagonists, especially quaternary ammonium muscarinic receptor antagonists such as ipratropium bromide, tiotropium bromide, and oxitropium bromide, as well as the salts, solvates, analogues, congeners, bioisosteres, hydrolysis products, metabolites, precursors, and prodrugs thereof.

In yet another alternative, the additional agent is a xanthine compound. The xanthine compounds especially preferred for use according to the invention include, but are not necessarily limited to, theophylline, extended-release theophylline, aminophylline, theobromine, enprofylline, diprophylline, isbufylline, choline theophyllinate, albifylline, arofylline, bamifylline and caffeine, as well as the salts, solvates, analogues, congeners, bioisosteres, hydrolysis products, metabolites, precursors, and prodrugs thereof.

In yet another alternative, the additional agent is an anti-IgE antibody. Typically, the anti-IgE antibody is a monoclonal antibody or a genetically engineered antibody that is derived from a monoclonal antibody. Preferably, the anti-IgE antibody is humanized. A particularly preferred humanized anti-IgE antibody is an IgG1 κ monoclonal antibody that specifically binds to human IgE and is marketed under the name of omalizumab.

In yet another alternative, the additional agent is a leukotriene modifier. The leukotriene modifiers especially preferred for use according to the present invention include, but are not necessarily limited to, ibudilast, montelukast, pranlukast, and zafirlukast, as well as the salts, solvates, analogues, congeners, bioisosteres, hydrolysis products, metabolites, precursors, and prodrugs thereof.

In yet another alternative, the additional agent is a phosphodiesterase IV inhibitor. The phosphodiesterase IV inhibitors especially preferred according to the present invention include, but are not necessarily limited to, roflumilast and cilomilast, as well as the salts, solvates, analogues, congeners, bioisosteres, hydrolysis products, metabolites, precursors, and prodrugs thereof. Phosphodiesterase IV is the predominant isoform in the lung and inhibitors of this enzyme are being considered for the treatment of asthma and COPD.

The route of administration of the β-adrenergic inverse agonist and of the additional therapeutic agent can be chosen by one of ordinary skill in the art to optimize therapeutic efficiency, as described above. However, in one preferred alterative, both the β-adrenergic inverse agonist and the additional therapeutic agent are administered by inhalation. In another preferred alterative, the β-adrenergic inverse agonist is administered orally, while the additional therapeutic agent is administered by inhalation. The administration of the additional therapeutic agent by inhalation is typically preferred because of possible toxicity of some of these additional therapeutic agents. However, other routes are possible.

Aerosol therapy allows an almost ideal benefit to risk ratio to be achieved because very small doses of inhaled medication provide optimal therapy with minimal adverse effects. A variety of additional therapeutic agents suitable for use in methods according to the present invention are available in aerosol formulation, including β₂-adrenergic agonists, steroids, and anticholinergics. However, the therapeutic efficiency of drugs administered by aerosolization depends not only on the pharmacological properties of the drugs themselves, but also on the characteristics of the delivery device. The characteristics of the delivery device influence the amount of drug deposited in the lungs and the pattern of drug distribution in the airways.

Aerosols are airborne suspensions of fine particles. The particles may be solids or liquids. Aerosol particles are heterodisperse (i.e. the particles are of a range of sizes) and aerosol particle size distribution is best described by a log normal distribution. Particles tend to settle (sediment), adhere to each other (coagulate), and adhere to structures such as tubing and mucosa (deposit). The particles delivered by aerosol can be conveniently characterized on the basis of their aerodynamic behavior. One parameter is the mass median aerodynamic diameter (MMAD). By definition, a particle distribution with an MMAD of 1 μM has the same average rate of settling as a droplet of unit density and 1 μM diameter.

The size of an aerosol particle, as well as variables affecting the respiratory system, influence the deposition of inhaled aerosols in the airways. On one hand, particles larger than 10 μM in diameter are unlikely to deposit in the lungs. However, particles smaller than 0.5 μM are likely to reach the alveoli or may be exhaled. Therefore, particles that have a diameter of between 1 μM and 5 μM are most efficiently deposited in the lower respiratory tract. Particles of these sizes are most efficient for the delivery of therapeutic agents for asthma.

The percentage of the aerosol mass contained within respirable droplets (i.e., droplets with a diameter smaller than 5 μM), depends on the inhalation device being used. Slow, steady inhalation increases the number of particles that penetrate the peripheral parts of the lungs. As the inhaled volume is increased, the aerosol can penetrate more peripherally into the bronchial tree. A period of breath-holding, on completion of inhalation, enables those particles that have penetrated to the lung periphery to settle into the airways via gravity. Increased inspiratory flow rates, typically observed in patients with acute asthma, result in increased losses of inhaled drug. This occurs because aerosol particles impact in the upper airway and at the bifurcations of the first few bronchial divisions. Other factors associated with pulmonary airway disease may also alter aerosol deposition. Airway obstruction and changes in the pulmonary parenchyma are often associated with pulmonary deposition in the peripheral airways in patients with asthma.

In aerosol administration, the nose efficiently traps particles before their deposition in the lung; therefore, mouth breathing of the aerosolized particles is preferred. The aerosolized particles are lost from many sites. Generally, the amount of the nebulized dose reaching the small airways is ≦15%. In many cases, approximately 90% of the inhaled dose is swallowed and then absorbed from the gastrointestinal tract. The small fraction of the dose that reaches the airways is also absorbed into the blood stream. The swallowed fraction of the dose is, therefore, absorbed and metabolized in the same way as an oral formulation, while the fraction of the dose that reaches the airways is absorbed into the blood stream and metabolized in the same way as an intravenous dose.

When drugs are administered topically (via aerosol delivery to the lungs), the desired therapeutic effects depend on local tissue concentrations, which may not be directly related to plasma drug concentrations. If a sufficiently large dosage of any drug is given, systemic activity can easily be demonstrated with any inhaled β₂-agonists or corticosteroid. This has several implications. First, for the selection of a drug to be inhaled, topical drugs must combine a high intrinsic activity within the target organ and rapid inactivation of the systemically absorbed drug. Secondly, fewer systemic adverse effects should be expected with drugs that have a low oral bioavailability (whether due to poor gastrointestinal absorption or high first-pass hepatic metabolism). Because most inhaled drugs are administered at a low dosage and have a low oral bioavailability, plasma concentrations of these drugs are much lower than after oral administration.

Furthermore, factors influencing pulmonary absorption should be considered. It was recently demonstrated that terbutaline was absorbed through the lung more rapidly in healthy smokers than in healthy nonsmokers. This may affect the onset of action of the drug. It has also been found that the bioavailability of inhaled salbutamol in 10 patients with cystic fibrosis was greater than that in healthy adults. One proposed mechanism for this difference in bioavailability is that the chronically diseased tracheobronchial tree in patients with cystic fibrosis results in higher permeability of salbutamol in this tissue. However, data are limited in this area, and further investiga-tion is required to substantiate these claims.

Finally, the absolute pulmonary bioavailability of inhaled drugs is difficult to assess because blood concentrations are low, and pulmonary and oral absorption should be discriminated for pulmonary bioavailability to be determined as accurately as possible. Charcoal can be used to adsorb the swallowed fraction of inhaled terbutaline to discriminate the pulmonary absorption of the drug. Recently, it was shown that a urine collection during the 30 minutes after inhalation of salbutamol represents the amount of drug delivered to the lungs. This technique may be applicable for the determination of bioavailability of other inhaled drugs. Other techniques for the determination of bioavailability of inhaled drugs are also known in the art; these include pharmacodynamic methods using FEV₁ measurements, lung deposition studies using radiolabeled formulations, or pharmacokinetic studies using predominantly urinary excretion measurements.

Therapeutic aerosols are commonly produced by atomization of liquids within jet nebulizers or by vibration of a standing pool of liquid (ultrasonic nebulisation). Preformed aerosols may also be administered. Examples of the latter include MDIs and dry powder devices. Whatever delivery device is used, patients should be taught to use it correctly.

All jet nebulizers work via a similar operating principle, represented by the familiar perfume atomizer. A liquid is placed at the bottom of a closed container, and the aerosol is generated by a jet of air from either a compressor or a compressed gas cylinder passing through the device. Ultrasonic nebulizers produce an aerosol by vibrating liquid lying above a transducer at frequencies of about 1 mHz. This produces a cloud of particles that is carried out of the device to the patient by a stream of air. Aerosols varying in quantity, size and distribution of panicles can be produced by nebulizers, depending upon the design of the nebulizer and how it is operated. It should be noted that not all nebulizers have the required specifications (MMAD, flow, output) to provide optimum efficacy. A recent study compared the lung deposition from 4 nebulizers in healthy volunteers and showed that median lung aerosol deposition, expressed as percentages of the doses initially loaded into the nebulizers, ranged from 2 to 19%. Nebulized aerosols are particularly useful for children under 5 years of age and in the treatment of severe asthma where respiratory insufficiency may impair inhalation from an MDI or dry powder inhaler. To minimize adverse effects, pH and osmolarity of the nebulized solution should be controlled.

Metered dose inhalers (MDIs), because of their convenience and effectiveness, are probably the most widely used therapeutic aerosol used for inhaled drug delivery to outpatients. Most MDIs in current use contain suspensions of drug in propellant. There are 2 major components of an MDI: (i) the canister, a closed plastic or metal cylinder that contains propellant, active medication, and the metering chamber; and (ii) the actuator, a molded plastic container that holds the canister and directs the released aerosol towards the patient's airway.

Propellant mixtures are selected to achieve the vapor pressure and spray characteristics desired for optimal drug delivery. Chlorofluorocarbons were previously used, but non-chlorinated propellants are now employed because of environmental concerns. Finely divided particles of drug, usually less than 1 μM, are suspended in the pressurized (liquified) propellant. To prevent the drug from coagulating, a surface active agent such as sorbitan oleate, lecithin or oleic acid is typically added; other surface active agents are known in the art. Metering chambers ordinarily contain 25 to 100 μL. The contents of the metering chamber are released when the canister is depressed into the actuator. Almost instantaneously, the propellants begin to evaporate, producing disintegration of the discharged liquid into particles that are propelled forward with great momentum. For optimal pulmonary drug deposition, the medication should be released at the beginning of a slow inspiration that lasts about 5 seconds and is followed by 10 seconds of breath-holding. Several inhalation aids have been designed to improve the effectiveness of a MDI. These are most useful in patients who have poor hand-to-breath coordination. A short tube (e.g. cones or spheres) may direct the aerosol straight into the mouth or collapsible bags may act as an aerosol reservoir holding particles in suspension for 3 to 5 seconds, during which time the patient can inhale the drug. However, when any of these devices is used, aerosol velocity upon entering the oropharynx is decreased and drug availability to the lungs and deposition in the oropharynx is decreased.

Dry powder inhalers have been devised to deliver agents to patients who have difficulty using an MDI (e.g. children and elderly patients). In general, the appropriate dosage is placed in a capsule along with a flow aid or filler such as large lactose or glucose panicles. Inside the device, the capsule is initially either pierced by needles (e.g. Spinhaler®) or sheared in half (e.g. Rotohaler®). During inhalation the capsule rotates or a propeller is turned, creating conditions that cause the contents of the capsule to enter the inspired air and be broken up to small particles suitable for delivery to the airways. The energy required to disperse the powder is derived from the patient's inspiratory effort. Recently, more convenient multidose dry powder inhalers have been introduced (e.g. Diskhaler® , Turbuhaler®). Potential problems associated with dry powder inhalers include esophageal irritation and, consequently, cough due to the direct effect of powder in airways. Furthermore, the walls of the capsule may be coated with drug as a result of either failure of the capsule to release the drug or failure of the aggregated powder to break up. This may cause virtually all of the drug to be deposited in the mouth. These powder devices do not contain chlorofluorocarbons and may provide an alternative to MDIs.

The clinical use of aerosols for asthma treatment has been proposed for several compounds proposed herein as additional therapeutic agents, including β₂-agonists and corticosteroids.

For β₂-agonists, limited pharmacokinetic data are available in humans mostly because the low dosages of inhaled drugs required for therapeutic activity produce drug concentrations in body fluids that are below assay limits. Little is known about pulmonary bioavailability of those drugs. It is generally argued that an average of 10% of an inhaled dose reaches the lung when given by a MDI. The mean pulmonary bioavailability of terbutaline from an MDI was reported to be 9.1%. When the oral component (swallowed fraction of the dose) was added, the value rose to 16.5%, i.e. an increase of 6.7%. The drugs salmeterol and formoterol have different mechanisms of action underlying their prolonged duration of bronchodilatory effect (12 to 18 hours). Salmeterol appears unique because it has a long side-chain that anchors the β₂-agonist molecule to the receptor. Formoterol appears to be an extremely potent classical β₂-agonist. The elimination half-life of formoterol after inhalation was calculated to be between 1.7 and 2.3 hours on the basis of urinary excretion data. A glucuronic acid conjugate was identified. However, it is possible that formoterol has a prolonged elimination half-life that is yet to be detected in humans. Salmeterol is formulated as the xinafoate (hydroxynaphthoic acid) salt. Little is known about the pharmacokinetic properties of this drug Salmeterol is extensively metabolized by hydroxylation, with the majority of a dose being eliminated predominantly in the feces within 72 hours. The hydroxynaphthoic acid part of the molecule accumulates in plasma during repeated administration as a consequence of its long elimination half-life (12 to 15 days).

For anticholinergic agents, the parent compound of this class is atropine. Synthetic agonists of the muscarinic receptors of acetylcholine are quaternary ammonium compounds and, therefore, cross membrane barriers with difficulty. Because systemic absorption of atropine after inhalation of the drug is nearly complete, this route of administration can produce significant systemic toxicity (Harrison et al. 1986). Ipratropium bromide is the only well studied representative of this class. Absorption through the gastrointestinal tract is slow, as peak plasma concentrations have been recorded 3 hours after oral intake of the drug. The absolute bioavailability after oral intake is only 30%. Elimination of metabolized drug occurs in the urine and bile. Whatever the route of administration, the mean elimination half-life is about 3.5 hours. Plasma concentrations observed with inhaled ipratropium were a thousand times lower than those observed with an equipotent bronchodilatory dose administered orally. This explains why systemic anticholinergic effects do not occur following inhalation of therapeutic doses of ipratropium bromide. These properties are probably shared by other quatemary ammonium anticholinergic agents such as oxitropium bromide, an alterative as described above.

Corticosteroids are frequently administered by inhalation, which can prevent some of the adverse effects usually associated with systemic corticosteroid therapy. To produce a compound with marked topical activity, some of the hydroxyl groups in the hydrocortisone molecule were substituted with acetonide or ester groups. Topically active corticosteroid drugs used for the treatment of patients with asthma include beclomethasone, betamethasone valerate, budesonide, triamcinolone, fluticasone and flunisolide. Of these, beclomethasone and budesonide are the most extensively used. The results of numerous clinical studies have shown that there is little difference between the efficacy of beclomethasone and budesonide. Oropharynx deposition is reduced by using a spacing device, and the development of candidiasis can be prevented by mouth rinsing. Plasma clearance of budesonide was calculated to be 84±27 L/h, which is about 10-fold higher than the average clearance of prednisolone. As a consequence of this high clearance, the elimination half-life of budesonide is short (2.8±1.1 hours). The systemic availability of the swallowed fraction is 10.7±4.3%, indicating that there is extensive first-pass metabolism. Stereoselective metabolism was demonstrated and plasma clearance of the 2 enantiomers, when calculated on a per kilogram of bodyweight basis, were about 50% higher in 6 children with asthma than in 11 healthy adults. Therefore, administration of budesonide by inhalation should reduce the risk of systemic adverse effects compared with administration of the drug orally. Lung esterases are known to hydrolyze beclomethasone. The absorbed beclomethasone and esterase-hydrolysis products (beclomethasone 17-propionate and beclomethasone) are rapidly converted to less active metabolites during passage through the liver. First-pass hepatic metabolism of the systemically absorbed fluticasone is almost complete, and therefore the inhaled drug has a favorable pharmacokinetic profile. Few data have been published regarding the pharmacokinetic properties of flunisolide, triamcinolone and betamethasone valerate.

To ensure maximal effects from inhaled drugs, both the pharmacological characteristics of the drugs and the device used to aerosolize the drugs should be considered. With respect to β₂-agonists, different formulations, with different pulmonary disposition techniques, are available, such as for MDI administration, for administration with a dry powder inhaler, or a solution for nebulization. A unit dose from a dry powder inhaler is twice that release from an MDI, but they have equivalent bronchodilatory effects. The characteristics of the devices vary. For a metered-dose inhaler, typically 1240% of the dose is deposited in the lung, but up to 80% in the oropharynx. When an MDI is used with a spacer, typically about 20% of the dose is deposited in the lung, but only up to 5% in the oropharynx; thus, the use of a spacer can reduce the proportion of the drug that is deposited in the oropharynx. For a dry powder inhaler, typically 11-16% of the dose is deposited in the lung and 31-72% in the oropharynx. For a nebulizer, typically 7-32% of the dose is deposited in the lung and 1-9% is deposited in the oropharynx. One of ordinary skill in the art can ensure that the proper inhalation therapy device is used and can prepare suitable instructions. Considerations for the use of inhalation therapy are described in A. M. Tabaret & B. Schmit, “Pharmacokinetic Optimisation of Asthma Treatment,” Clin. Pharmacokinet. 26: 396-418 (1994), incorporated herein by this reference.

For all of these combinations, the invention further encompasses blister packs that contain either a fixed-dose combination of the inverse agonist for the GPCR and the additional therapeutic agent, such as the β₂-selective adrenergic agonist, the steroid, the anticholinergic agent, the xanthine compound, the anti-IgE antibody, the leukotriene modifier, or the phosphodiesterase IV inhibitor, or, in separate pills, capsules, or other dosage forms, the inverse agonist for the GPCR and the additional therapeutic agent. The use of these blister packs is appropriate when oral administration of the inverse agonist and additional therapeutic agent is desired. The blister packs follow the general design described above and in FIG. 1, and include appropriate instructions to the patient.

In general, when a fixed-dose combination is used, the blister pack comprises:

(1) a lower substrate;

(2) an intermediate dosage holder that is shaped to generate a plurality of cavities and that is placed over the lower substrate, the cavities being shaped to hold dosage forms of the pharmaceutical composition described above containing the inverse agonist and an additional therapeutic agent;

(3) an upper substrate placed over the intermediate dosage holder that has a plurality of apertures, each aperture being located to accommodate a corresponding cavity; and

(4) dosage forms of the pharmaceutical composition placed in the cavities.

When the the β-adrenergic inverse agonist and the additional therapeutic agent are to be administered in separate dosage forms, the blister pack, in general, comprises:

(1) a lower substrate;

(2) an intermediate dosage holder that is shaped to generate a plurality of cavities and that is placed over the lower substrate, the cavities being shaped to hold dosage forms of: (a) a first pharmaceutical composition that comprises: (i) a therapeutically effective amount of the inverse agonist; and (ii) a first pharmaceutically acceptable carrier; and (b) a second pharmaceutical composition that comprises: (i) a therapeutically effective amount of a second therapeutic agent as described above, the second therapeutic agent being selected from the group consisting of a β₂-selective adrenergic agonist, a steroid, an anticholinergic drug, a xanthine compound, an anti-IgE antibody, a leukotriene modifier, and a phosphodiesterase IV inhibitor; and (ii) a second pharmaceutically acceptable carrier;

(3) an upper substrate placed over the intermediate dosage holder that has a plurality of apertures, each aperture being located to accommodate a corresponding cavity; and

(4) dosage forms of the first and second pharmaceutical compositions placed in the cavities.

The dosage forms of the first and second pharmaceutical compositions are as described above. Typically, in this arrangement, the dosage forms of the first pharmaceutical composition include dosages starting at a low dose and including a range of dosages up to the highest, maintenance, dose. Other dosage form arrangements are possible.

Other arrangements are possible for the blister packs.

In another alternative, the disease or condition associated with the activity of a GPCR can be congestive heart failure (CHF). When the disease or condition associated with the activity of a GPCR is CHF, the GPCRs are also β₂-adrenergic receptors, and the inverse agonist is typically nadolol.

Methods according to the present invention can also be used for the treatment of diseases and conditions associated with the activity of other GPCRs, including, but not limited to, acetylcholine receptors (including muscarinic receptors), α-adrenergic receptors, β₃-adrenergic receptors, serotonin (5-hydroxytryptamine) receptors, dopamine receptors, adenosine receptors, angiotensin Type II receptors, bradykinin receptors, calcitonin receptors, calcitonin gene-related receptors, cannabinoid receptors, cholecystokinin receptors, chemokine receptors, cytokine receptors, gastrin receptors, endothelin receptors, γ-aminobutyric acid (GABA) receptors, galanin receptors, glucagon receptors, glutamate receptors, luteinizing hormone receptors, choriogonadotrophin receptors, follicle-stimulating hormone receptors, thyroid-stimulating hormone receptors, gonadotrophin-releasing hormone receptors, leukotriene receptors, Neuropeptide Y receptors, opioid receptors (Lesscher et al., Eur. J. Neurosci. 17: 1006-1012 (2003)), parathyroid hormone receptors, platelet activating factor receptors, prostanoid (prostaglandin) receptors, somatostatin receptors, thyrotropin-releasing hormone receptors, vasopressin and oxytocin receptors, and other physiologically active receptors.

In particular, diseases and conditions associated with the activity of the opioid receptors are significant. The use of inverse agonists together with agonists to these receptors can allow pain management without the tolerance associated with opioid-mediated down-regulation of opioid receptors and the ability of opioid antagonists to prevent opioid receptor internalization (S. M. Crain & K. F. Shen, “Ultra-Low Concentrations of Naloxone Selectively Antagonize Excitatory Effects of Morphine on Sensory Neurons, Thereby Increasing Its Antinociceptive Potency and Attenuating Tolerance/Dependence During Chronic Cotreatment,” Proc. Natl. Acad. Sci. 92: 10540-10544 (1995); A. Tempel et al., “Morphine-Induced Downregulation of μ-Opioid Receptors in Neonatal Rat Brain,” Brain Res. 469: 129-133 (1988); N. Marie et al., “Differential Sorting of Human δ-Opioid Receptors After Internalization by Peptide and Alkaloid Antagonists,” J. Biol. Chem. 278: 22795-22804 (2003); C. N. Patel et al., “Chronic Opioid Antagonist Treatment Selectively Regulates Trafficking and Signaling Proteins in Mouse Spinal Cord,” Synapse 50: 67-76 (2003)). This can provide improved pain management.

Also in particular, diseases and conditions associated with the activity of the β₃-adrenergic receptor are significant. These diseases and conditions include obesity. The medical consequences of obesity in the developed countries, especially the United States, are severe and are increasing in frequency. These consequences include adult-onset diabetes, cancer, and cardiovascular diseases. Obesity, because of the stress that it places on the body, can also worsen musculoskeletal disorders such as arthritis. It is also suspected as having an association with neurological conditions such as dementia. The usual causes cited are diet and a lack of exercise. Thus, improved methods of treating or preventing obesity are extremely important.

The β₃-adrenergic receptor was identified pharmacologically in the 1980's (B. J. Lipworth, “Clinical Pharmacology of β₃-Ardenoceptors,” Br. J. Clin. Pharmacol. 42: 291-300 (1996)), and cloned in 1989 (L. J. Emorine et al., “Molecular Characterization of the Human β₃-Adrenergic Receptor,” Science 245: 1118-1121(1989)). β₃-agonists preferentially stimulate lipolysis in white and brown adipose tissue (WAT and BAT). β₃ receptor gene polymorphisms are associated with obesity in Pima Indians and other groups. In addition, the β₃ receptor mRNA is downregulated in two genetically obese rodent models, obese ob/ob mice and fa/fa rats (A. D. Strosberg & F. Pietri-Rouxel, “Function and Regulation of the β₃-Adrenoceptor, “Trends Pharmacol. Sci. 17: 373-381 (1996)). Though the single murine knockout β₃ receptor does not result in obesity, a triple knockout of the β₁/β₂/β₃ receptors results in massively obese mice when fed a high fat diet, demonstrating functional redundancy (E. S. Bachman et al., βAR Signaling Required for Diet-induced Thermogenesis and Obesity Resistance,” Science 297: 843-845 (2002)). Consequently, the β₃ receptor is viewed as a validated target for obesity.

β₃ agonists have not progressed further than Phase II (J. R. Arch, β(3)-Adrenoceptor Agonists: Potential, Pitfalls and Progress, Eur. J. Pharmacol. 440: 99-107 (2002)). This is due in part to the fact that mouse and rodent obesity models and their β₃ receptors are substantially different than humans (J. R. Arch & S. Wilson, β₃-Adrenoceptors and the Regulation of Metabolism in Adipose Tissues,” Biochem. Soc. Trans. 24: 412418 (1996); J. R. Arch & S. Wilson, “Prospects for β₃-Adrenoceptor Agonists in the Treatment of Obesity and Diabetes,” Int. J. Obes. Relat. Metab. Disord. 20: 191-199 (1996)). In addition, selectivity for the β₃ receptor is needed to avoid serious agonist side effects at the β₁/β₂ receptors. Ephedrine, a non-selective beta agonist, has been used over-the-counter as a diet drug and is known to be active at all three β receptors. The use of ephedrine has led to serious adverse events, including heart failure, due to its non-selectivity; its use has led to several highly publicized deaths, including that of at least one professional athlete in his mid-twenties. Thus, the benefits of non-selective beta agonists for obesity do not outweigh the risks. In addition, β₃ receptors are not only expressed in white and brown adipocytes but are present in the heart muscle, skeletal muscle, bladder smooth muscle, gastrointestinal tract, and gall bladder. Thus, even if a selective β₃ agonist is developed, it may still result in serious side effects due to the widespread distribution of the receptor.

A less obvious reason for failure of β₃ agonists in clinical trials may be reflected by the unpublished observations on the β₃ agonist L-796568 in healthy volunteers: “the acute increases (in plasma fatty acid concentrations) measured on the first treatment day were completely absent after 9 d of treatment (from T. M. Larsen et al., “Effect of a 28-d Treatment with -796568, a Novel β(3)-Adrenergic Receptor Agonist, on Energy Expenditure and Body Composition in Obese Men,” Am. J. Clin. Nutr. 76: 780-788 (2002), referring to K. Gottesdiener and Merck & Co., unpublished observations, 1999)). This statement refers to the development of tolerance to the agonist drug, a phenomenon observed with GPCR agonist drugs in general. In contrast to humans, rodents have not been observed to develop tolerance to β agonists which may reflect differences in physiology, fat deposits (WAT versus BAT), and life history of rodents versus humans.

In humans, adipocytes express all three β adrenergic receptors (J. Hoffstedt et al., “Determination of β₃-Adrenoceptor Mediated Lipolysis in Human Fat Cells,” Obes. Res. 3: 447457 (1995)) and the α₁ receptor. The demonstration in mice of functional redundancy of the receptors and the finding that, in humans, ephedrine, in the presence of nadolol, results in a level of thermogenesis that is 40% of the ephedrine-untreated level, both demonstrate that the β₃ receptor is not solely responsible for lipolysis and that the Pi and β₂ receptors contribute as well.

Based on the rule elucidated above that chronic treatment with GPCR inverse agonists is equivalent to acute treatment with GPCR agonists, Applicant predicts that an inverse agonist targeting the β₃ receptor, or, more preferably targeting all three functionally redundant β receptors, would be an effective approach to increase lipolysis and ultimately reduce obesity.

Treatment with inverse agonists results in several effects, most obviously, upregulation of GPCR receptors. Overexpression of the β₁ receptor in transgenic mice led to their partial resistance to obesity (V. Soloveva et al., “Transgenic Mice Overexpressing the β₁-Adrenergic Receptor in Adipose Tissue, Mol. Endocrinol. 11: 27-38 (1997), suggesting that β receptor upregulation via inverse agonist administration can be a sufficient treatment for this chronic health problem.

An additional issue may be that there may not be sufficient spontaneous activity of the β₃ receptor. If so, then enhancement of activity in the presence of upregulated receptor levels may be achieved by the endogenous agonist (norepinephrine) or the supplementation with exogenous β₃ agonists, For example, in obese humans WAT may not be sufficiently innervated to supply sufficient levels of endogenous agonist to the adipocytes, thus necessitating exogenous supplementation.

One unknown is whether it is preferred to use a selective β₃ inverse agonist that has no activity on the other two receptors (β₁ and β₂) or to use a nonselective inverse agonist. Very few β₃ inverse agonists have been identified, primarily as a byproduct in the search for β₃ agonists. Nevertheless, Saccomanni et al. (2003).(G. Saccomanni et al., “Synthesis and Beta-Blocking Activity of (R,S)-(E)-Oximeethers of 2,3-Dihydro-1,8-Naphthyridine and 2,3-Dihydrothiopyrano[2,3-b]pyridine: Identification of β₃-Antagonists,” Bioorg. Med. Chem. 11: 4921-4931 (2003)) identified several β₃ selective antagonists and SR 59230A (L. Manara et al., “β₃-Adrenoceptors and Intestinal Motility,” Fundam. Clin. Pharmacol. 9: 332-342 (1995); L. Manara et al., “Functional Identification of Rat Atypical β-Adrenoceptors by the First β₃-Selective Antagonists, Aryloxypropanolaminotetralins,” “Br. J. Pharmacol. 117: 435-442 (1996); available from Tocris), is a selective β₃ antagonist.

Alternatively, it may be preferable to use a nonselective β₃ inverse agonist since human adipocytes express all three receptors. The receptors are functionally redundant and may exhibit “crosstalk.” One possibility is bupranolol that has K₁ values of 1.7 nm for β₂, 0.4 nm for β₂, and 50 nm for β₃. Bupranolol was approved for hypertension and marketed in Europe under the name Betadrenol and was developed for glaucoma under the name Ophtorenin. However, the drug has a very short half-life (1.5 hours) and thus may require further formulation work to ensure long-term occupancy on the receptor to achieve the required degree of compensatory receptor upregulation. Towards this goal, Kemken et al. (J. Kemken et al., “Pharmacodynamic Effects of Transdermal Bupranolol and Timolol in Vivo: Comparison of Microemulsions and Matrix Patches as Vehicle,” Methods. Find. Exp. Clin. Pharmacol. 13: 361-365 (1991)) worked on developing a transdermal patch which was not developed further. Saccomanni et al. (2003), supra, also identified non-selective antagonists as well that may prove useful.

Therefore, another aspect of the invention is a method for the treatment or prevention of obesity comprising administering a therapeutically effective quantity of a β₃ adrenergic inverse agonist to treat or prevent obesity. The inverse agonist can be selective for β₃ or can be non-selective, having inverse agonist activity for β₁ and/or β₂ in addition to β₃. The inverse agonist can be administered in a timed-release formulation to ensure receptor occupancy. An example of a suitable β₃ adrenergic receptor inverse agonist is bupranolol, as described above. As detailed below, the inverse agonist can be administered with a β₃ adrenergic receptor agonist in combination therapy. The β₃ adrenergic receptor agonist can be selective or non-selective. An example of a β₃ adrenergic receptor agonist is carazolol (A. Mejean et al., “Carazolol: A Potent, Selective β₃-Adrenoceptor Agonist,” Eur. J. Pharmacol. 291: 359-366 (1995)).

These methods can also further comprise the administration of an appropriate agonist to the GPCR, as detailed below.

Methods according to the present invention can further be used for the treatment of diseases and conditions associated with GPCRs disclosed in G. Milligan & R. A. Bond, “Inverse Agonism and the Regulation of Receptor Number,” Trends Pharmacol. Sci. 12: 468-474 (1997), incorporated herein by this reference, and in R. A. Bond, “Is Paradoxical Pharmacology a Strategy Worth Pursuing?,” Trends Pharmacol. Sci. 22: 273-276 (2001), also incorporated herein by this reference.

Methods according to the present invention can be used in human patients. Alternatively, methods according to the present invention can be used in socially or economically important animals such as dogs, cats, cattle, sheep, pigs, goats, and horses.

Another aspect of the present invention is a screening method for detecting active agents that are inverse agonists and that are capable of treating diseases and conditions associated with the activity of G protein coupled receptors. In general, such a screening method comprises:

(1) providing a population of specific G protein coupled receptors characterized by a constitutive basal level of activity in the absence of an agonist;

(2) contacting the population of specific G protein coupled receptors with a compound to be screened for its inverse agonist activity, the compound not being an agonist of the population of specific G protein coupled receptors; and

(3) determining the constitutive basal level of activity of the specific G protein coupled receptors in the absence of the compound and in the presence of the compound, such that the constitutive basal level of activity decreases if the compound is an inverse agonist.

In this method, the compounds chosen to be screened can be selected by a number of techniques generally known in the art of drug discovery and screening. These techniques include the use of naturally-occurring compounds, typically isolated from plants, animals, bacteria, or fungi, as well as the discovery and modification of lead compounds. The discovery of lead compounds can be carried out by random screening or targeted screening. The modification of lead compounds can be carried out through various principles of pharmacological chemistry, including identification of the pharmacophore, functional group modification, determination of structure-activity relationships, preparation of homologues, modifications in chain branching in alkyl groups, ring-chain transformations, and identification of bioisosteres, The modification and selection of compounds chosen to be screened can be carried out by conventional chemical techniques or through solid-phase approaches, typically using parallel synthesis techniques. In the parallel synthesis approach, a suitable structure is chosen as the scaffold, and this can be based on an important molecular recognition motif in the target receptor. The reactions are carried out individually in separate microtubes, applying automation. These techniques are described in R. B. Silverman, “The Organic Chemistry of Drug Design and Drug Action” (2^{nd} ed., 2004, Elsevier, Amsterdam), ch. 2, pp. 7-120.

The constitutive basal level of activity of the specific G protein coupled receptors in the absence and in the presence of the compound can be measured by various techniques, depending on whether intact organisms, cell cultures, or tissue cultures are being used. For example, the production or activity of a second messenger such as cyclic AMP (cAMP) can be measured. If intact organisms are used, the physiological consequences of receptor activation, such as airway resistance, can be measured. In many systems, it is desirable to transform or transfect cells with genetically engineered constitutively active mutant receptors (CAM). This can be done by standard genetic engineering techniques. Alternatively, overexpression of the wild-type receptors can be induced. These approaches are described in R. A. F. de Ligt et al., “Inverse Agonism at G Protein-Coupled Receptors: (Patho)physiological Relevance and Implications for Drug Discovery,” Br. J. Pharmacol. 130: 1-12 (2000), incorporated herein by this reference.

This screening method can be used to detect active agents that are inverse agonists for acetylcholine receptors, including muscarinic receptors, β₂-adrenergic receptors, β₃-adrenergic receptors, and other receptors described above. Therefore, this screening method can be used to detect agents that can be used to treat diseases and conditions associated with these receptors, including, but not limited to, pulmonary airway diseases, including asthma, chronic allergic rhinitis, and other diseases and conditions associated with hypofunction of these receptors.

Another screening method according to the present invention relies on the finding, described above, that exposure of cells containing a specific population of G protein coupled receptors to an inverse agonist for a substantial period of time results in an increase in receptor population or receptor density in the cells. Therefore, this alternative of the screening method comprises:

(1) providing cells containing a population of specific G protein coupled receptors characterized by a constitutive basal level of activity in the absence of an agonist;

(2) contacting the cells containing the population of specific G protein coupled receptors with a compound to be screened for its inverse agonist activity, the compound not being an agonist of the population of specific G protein coupled receptors, the compound being contacted with the cells for a period of time to result in an increase in receptor population or receptor density if the compound is an inverse agonist; and

(3) determining the receptor population or receptor density of the specific G protein coupled receptors in the cells in the absence of the compound and in the presence of the compound, such that the receptor population or receptor density increases if the compound is an inverse agonist.

The receptor population or receptor density can be determined by an immunochemical method, using labeled antibodies specific for the receptor, although other methods can be used. The use of such labeled antibodies is well known in the art and need not be described further here; radioactive labels or fluorescent labels can be used. This is used in Example 3, below. Alternatively, the receptor population or receptor density can be determined by the binding of a radioligand with an affinity sufficiently high to bind all receptors and measuring the extent of binding. This is used in Example 2, below.

This screening method can also used to detect active agents that are inverse agonists for acetylcholine receptors, including muscarinic receptors, β₂-adrenergic receptors, β₃-adrenergic receptors, and other receptors described above. Therefore, this screening method can also be used to detect agents that can be used to treat diseases and conditions associated with these receptors, including, but not limited to, pulmonary airway diseases, including asthma and chronic allergic rhinitis, as well as other diseases and conditions associated with hypofunction of GPCRs.

These treatment and screening methods are significant. GPCRs account for 60% of drug targets on market, e.g. beta agonists, alpha blockers, beta blockers, and other targets. Drug discovery for GPCR targets has generally been focused on “acute” effects at protein, cellular level and in animals. Physicians and scientists have generally extrapolated that chronic effects of drug would be identical to acute drug effects. However, the present invention has shown that the acute effects of many drugs do not equal their chronic effects, and that this can be exploited to identify new drug therapeutics. This is a general, previously unrecognized phenomenon that explains paradoxical benefit effects of drugs in various therapeutic indications. The present invention demonstrates this for beta inverse agonist use in asthma and explains the efficacy of this drug class in CHF as not being an isolated example as most in the field view it.

For CHF the acute effects of beta agonists do not equal their chronic effects; beta agonists helpful acutely but increase mortality chronically. The serendipitous discovery that beta blockers have huge benefit in reducing mortality upon chronic administration despite short-term acute detriment has been generally viewed as a single isolated incidence of inherent paradox. However, the present invention makes clear that a novel route of drug action and a novel method of drug discovery underlie this seemingly isolated finding.

The present invention makes clear that that there are many examples of GPCRs that are up-regulated by inverse agonists. These observations have not been completely valued till now. The present invention also demonstrates that GPCRs (for example, β₂-adrenoceptors in transgenic mouse) are spontaneously active in absence of agonist.

Whilst not being held to this theory, the inventor believes that part of the explanation for the therapeutic effect of chronic administration of inverse agonists is the up-regulation of spontaneously active GPCRs, this may also include upregulation of internal components of signal transduction pathway.

The present invention also incorporates the finding, first in vitro then in vivo, that GPCRs have activity in the absence of ligand. This new appreciation of GPCRs impacts our understanding that there are three classes of drugs that can interact with two different forms of a GPCR.

The invention is illustrated by the following Examples. These Examples are for illustrative purposes only and are not intended to limit the invention.

### EXAMPLES

### Example 1

### Airway Resistance Reduction by Chronic Administration of β-Adrenergic Inverse Agonists

Methods

Balb/cJ mice aged 6 weeks (Jackson Animal Laboratory, Bar Harbor, Maine) were housed under specific pathogen-free conditions and fed a chicken ovalbumin-free diet. The Animal Research Ethics Boards of both the University of Houston and the Baylor College of Medicine approved all experiments reported here. The effects of administration of the non-selective β-adrenergic inverse agonists carvedilol (GlaxoSmithKline, King of Prussia, Pa.) and nadolol (Sigma Chemical, St. Louis, Mo.) and of salbutamol (Sigma Chemical, St. Louis, Mo.), a β₂-adrenergic partial agonist, were examined in a murine model that exhibited cardinal features of human asthma, such as pulmonary eosinophilic inflammation, airway hyperresponsiveness, and heterogenous airway narrowing. The results obtained in drug-treated animals were compared with those obtained in vehicle-treated counterparts (controls) in experiments performed in close temporal relationship. The outcome measures of the study of Example I included statistically-significant differences between drug-treated mice and non-treated animals in terms of baseline airway resistance, degree of airway responsiveness to cholinergic stimulation, and bronchoalveolar lavage (BALF) cellularity. Mice were sensitized with subcutaneous injection of 25 μg of ovalbumin adsorbed to aluminum hydroxide on protocol days 2, 9, and 16. Subsequently, mice were given 50 μL of saline solution containing 25 μg of ovalbumin intranasally, on a daily basis, from protocol days 23 through 27. A group of ovalbumin-sensitized saline-challenged mice serves as controls for systemic sensitization and respiratory challenges with ovalbumin. Prior to intranasal administrations, mice were sedated with halothane vapor. For the study of Example 1, ovalbumin-sensitized and ovalbumin-challenged mice, and ovalbumin-sensitized and saline-challenged mice will be referred to as asthmatic mice and control mice, respectively. The drugs used were salbutamol (a β₁/β₂-adrenergic agonist), alprenolol (a β₁/β₂-adrenergic antagonist with partial agonist activity), and nadolol and carvedilol (both non-selective β₁/β₂ adrenergic inverse agonists).

To examine the effects of duration of β-adrenergic ligand therapy on the phenotype of the murine model of asthma, the experimental drugs were administered either acutely or chronically to different groups of asthmatic mice.

Asthmatic mice on acute therapy were given a single intravenous bolus infusion of either β-adrenergic drug or normal saline on protocol day 28, 15 minutes before airway responsiveness to methacholine was determined. The doses of carvedilol, nadolol, alprenolol, and salbutamol administered to the mice were 24 mg/kg, 72 mg/kg, 72 mg/kg, and 0.15 mg/kg, respectively. Asthmatic mice on chronic therapy were treated with the β-adrenergic drug during protocol days 1 to 28. Those on β-antagonists had free access to chow treated with carvedilol, nadolol, or alprenolol at concentrations of 2400 ppm, 250 ppm, or 7200 ppm, respectively. These concentrations were chosen based on those producing therapeutic effects in mice in previously published studies. The non-asthmatic mice were fed normal chow. Salbutamol was delivered for 28 days at a dose of 0.5 mg/kg/day using an osmotic minipump (Alzet®, #2004, Durect Corporation, Cupertino, Calif.).

On protocol day 28, mice were anesthetized, tracheotomized, and connected to a computer-controlled small animal ventilator (Flexivent, Scientific Respiratory Equipment, Inc., Montreal, Canada). Airway resistance (R_{aw}) was measured using the forced oscillation technique. The cellular composition of bronchoalveolar lavage fluid (BALF) was also determined. In non-treated asthmatic mice, the degree of airway responsiveness and the number of eosinophils recovered in BALF were significantly higher compared to the ovalbumin-sensitized saline-challenged (control) mice. However, it was observed that the degree of airway responsiveness and the number of eosinophils recovered in BALF were lower in non-treated asthmatic mice studied in close temporal relationship with mice receiving acute β-adrenergic antagonist treatments that in those obtained in non-treated asthmatic mice studied concomitantly with mice on chronic β-adrenergic antagonist therapy.

To induce airway constriction, a solution containing 150 μg/mL of acetyl-α-methylcholine chloride (methacholine) (Sigma Chemical, St. Louis, Mo.) was infused intravenously at constant rates using a syringe infusion pump (Raze Scientific Instruments, Stanford, Conn.). The methacholine infusion was started at 0.008 mL/min, and its rate was doubled stepwise up to a maximum of 0.136 mL/min. Each methacholine dose was administered for 3 to 5 minutes, during which data were sampled at 1-minute intervals and then averaged.

Data Analysis

The complex input impedance of the respiratory system was computed and the value of the real part of respiratory system impedance at 19.75 Hz was taken to reflect the magnitude of airway resistance (R_{aw}). To examine the degree of airway responsiveness of each animal, the values for R_{aw} as a function of methacholine doses were plotted. The largest value for R_{aw} achieved in response to methacholine stimulation was referred to as R_{awpeak}. For mice that achieved a plateau in the methacholine dose-R_{aw} response curve, the ED₅₀ was calculated by linear interpolation using the GraphPad Prism4 (GraphPad Software, Inc.). Results obtained for β-adrenergic drug treated and non-treated mice were performed using the analysis of variance for multiple groups of a Student's t-test for comparing two groups. The Bonferroni test was used to examine the statistical differences between experimental groups. The effects of acute drug treatments on baseline respiratory system mechanics were assessed using a two-tailed paired t-test. A value of P<0.05 was considered significant.

FIG. 2

FIGS. 2A and 2B show that methacholine provocation significantly enhances airway resistance (R_{aw}) in asthmatic mice in contrast to a minimal response upon saline provocation of asthmatic mice. This demonstrates that the mouse model in this study exhibits airway hyperresponsiveness, a key feature of airway dysfunction in human asthma.

In FIG. 2C, the administration of a single intravenous bolus of salbutamol to asthmatic mice reduced the level of airway responsiveness to methacholine provocation and the level of airway resistance as expected, thus demonstrating an acute effect of this agent. However, in FIG. 2D, when salbutamol was delivered for 28 days to the mice, no protection was observed. This lack of reduction of airway hyperresponsiveness upon chronic administration of a β-adrenergic agonist has been observed in humans when tolerance to these drugs develop.

In FIG. 2E, when asthmatic mice were given a single intravenous bolus of alprenolol, a β-adrenergic antagonist with partial agonist activity, their airway responsiveness was diminished, as indicated by significant decreases in both the values for R_{aw} at methacholine doses ≧408 μg/kg/min (P<0.05) compared with those obtained in non-treated counterparts. The reduction in airway responsiveness upon acute administration of alprenolol is similar to that observed for salbutamol, consistent with the partial agonist activity that alprenolol possesses. In FIG. 2F, when asthmatic mice were exposed to alprenolol for 28 days, their average methacholine dose-response relationship was similar to that obtained in nontreated mice, demonstrating that this drug provides no benefit upon chronic administration, as is the case with salbutamol. This is again directly analogous to the tolerance seen in human patients after long-term administration of such drugs.

In FIG. 2G, a single intravenous bolus of carvedilol enhanced the airway responsiveness in the asthmatic mice. This is consistent with previous observations in humans that acute delivery of β-adrenergic antagonists to asthmatics can result in severe airway constriction. In contrast, in FIG. 2H, chronic administration of carvedilol reduced the responsiveness of asthmatic mice to methacholine provocation. Chronic delivery of carvedilol not only decreased the airway constrictor response at high doses of methacholine, it also shifted the methacholine dose-airway response relationship to the left of that obtained in the non-treated asthmatic mice.

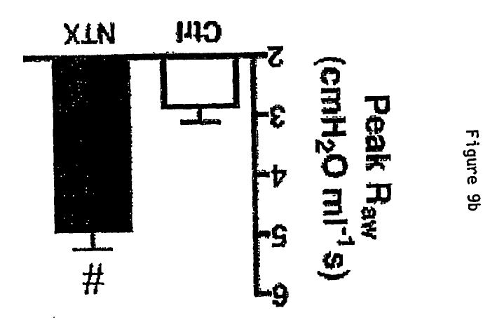

In FIG. 21, a single intravenous bolus of nadolol also enhanced the airway responsiveness of asthmatic mice similar to that observed for carvedilol. Chronic delivery of nadolol, as shown in FIG. 2J, also produced a decrease in airway responsiveness, which was more pronounced than that caused by carvedilol treatment. Indeed, the average methacholine dose-R_{aw} response relationship obtained in asthmatic mice on chronic nadolol treatment was similar to that obtained in mice on acute salbutamol treatment.

FIG. 3

FIG. 3 shows the effects of administration of β-adrenergic receptor ligands on the peak airway responsiveness to cholinergic stimulation in asthmatic mice. Peak R_{aw} was determined for each mouse by examining the individual methacholine dose-response curves and choosing the highest R_{aw} value produced by any of the methacholine doses (most often the next to last dose, 408 μg kg⁻¹ min⁻¹). Shown are the mean peak R_{aw}±SEM after treatments with the β-adrenergic receptor agonist salbutamol (A), after acute treatments with various agents (B) (ALP=alprenolol; CAR=carvedilol; NAD; nadolol); and after chronic treatments with the same agents used in (B), all in comparison to nontreated asthmatic mice (NTX) (black bars, n=7-25) and control mice (Ctrl, white bars, n=6-21). Values are mean±SEM for the peak R_{aw} values to methacholine of n=8-19 mice. Note the change in scale of the y-axis for (B). *, P<0.05 compared to NTX; #, P<0.05 compared to Ctrl (ANOVA).

FIG. 3

FIG. 3 shows the effects of administration of β-adrenergic receptor ligands on the peak airway responsiveness to cholinergic stimulation in asthmatic mice. Peak _{aw} was determined for each mouse by examining the individual methacholine dose-response curves and choosing the highest R_{aw} value produced by any of the methacholine doses (most often the next to last dose, 408 μg kg⁻¹ min⁻¹). Shown are the mean peak R_{aw}±SEM after treatments with the β-adrenergic receptor agonist salbutamol (A), after acute treatments with various agents (B) (ALP=alprenolol; CAR=carvedilol; NAD; nadolol); and after chronic treatments with the same agents used in (B), all in comparison to nontreated asthmatic mice (NTX) (black bars, n=7-25) and control mice (Ctrl, white bars, n=6-21). Values are mean±SEM for the peak R_{aw} values to methacholine of n=8-19 mice. Note the change in scale of the y-axis for (B). *, P<0.05 compared to NTX; #, P<0.05 compared to CtrI (ANOVA).

### Example 2

### Chronic Inverse Agonist Treatment Increases β-Adrenergic Receptor Numbers as Measured by Radioligand Binding

β₂-adrenergic receptor numbers were measured in asthmatic mice as follows. Asthmatic mice (ovalbumin-challenged) were treated as follows: Ctrl, no drug treatment with methacholine challenge; salbutamol, a short-acting β₂ agonist; carvedilol, a β₁, β₂ non-selective inverse agonist with α₁-adrenergic antagonist activity; nadolol, a highly specific, hydrophilic β₁, β₂ non-selective inverse agonist; and alprenolol, a β-adrenergic antagonist. Drug treatments were either a single treatment 15 minutes prior to methacholine challenge or ongoing for 28 days (salbutamol was delivered continuously via a subcutaneous osmotic minipump and alprenolol, carvedilol, and nadolol were in animal chow). Mice were sacrificed and lung membranes were isolated as follows. Frozen lung tissue was homogenized in an ice-cold buffer containing 0.32 M sucrose and 25 mM Tris (pH 7.4) using a polytron (Pro 200, Pro Scientific, Inc.). The homogenate was centrifuged at 1000×g for 10 min at 4° C. The resulting supernatant was centrifuged at 40,000×g for 20 min at 4° C. The pellet was suspended in an ice-cold 25 mM Tris-HCl buffer (pH 7.4) and centrifuged at 40,000'g for 20 min at 4° C. The final pellet was suspended in 200 μL of 25 mM Tris-HCl (pH 7.4); membrane protein concentration was determined by BCA protein assay kit. Radioligand receptor binding incubation mixtures contained membranes (˜10 μg of protein), (−)3-[¹²⁵I]-cyanopindolol (ICYP) in 25 mM Tris-HCl, pH 7.4, in increasing concentrations (5-7500 pM) and binding buffer in a final volume of 250 μL. Propranolol was used to determine nonspecific binding. The incubation was done at 37° C. for 2 h and terminated by rapid vacuum filtration through glass fiber filters. The filters were washed three times with 250 μL of cold wash buffer (25 mM Tris-HCl, pH 7.4) and the radioactivity determined in a counter. All experiments were performed in triplicate and values are mean±SEM of n=3-5 animals in each group. Receptor densities are expressed as femtomoles of sites per milligram of protein. Bₘₐₓ is determined by nonlinear regression of the saturation binding curves. Apparent K_{D} values (in parentheses) are expressed as pM. Please note the 15 min and 28 day tome points refer to duration of drug treatment. All mice were killed at the same age and thus for vehicle treated groups (Ctrl and NTX) the groups were identical and the results pooled. #P<0.05 compared to Ctrl; *P<0.05 compared to NTX (Student's t-test).

Radioligand binding revealed that β₂-adrenergic receptor levels appear to be somewhat lower in methacholine-challenged but otherwise untreated asthmatic mice as compared with untreated, unchallenged mice, as shown in Table 1. Chronic alprenolol treatment led to a slight decrease of the level of the β₂-adrenergic receptor. The same was true of chronic salbutamol treatment. Most significantly, the carvedilol-treated mice demonstrated an over 10-fold increase of the level of β₂-adrenergic receptors over the non-treated mice, demonstrating the efficacy of this β-adrenergic inverse agonist in increasing receptor levels upon chronic administration. Similarly, the nadolol-treated mice demonstrated a nearly eightfold increase of the level of receptors over the untreated methacholine-challenged asthmatic mice.

TABLE 1
Determination of β-Adrenergic Receptor Density by Radioligand Binding

| | 15 Minutes | | 28 Days | |
|---|---|---|---|---|
| Treatment | Bₘₐₓ | K_{D} | βₘₐₓ | K_{D} |
| Ctrl | 286.8 ± 88.02 | (107.9 ± 43.67) | 286.8 ± 88.02 | (107.9 ± 43.67) |
| NTX | 109.2 ± 9.72# | (193.6 ± 20.66) | 109.2 ± 9.72# | (193.6 ± 20.66) |
| Salbutamol | 256.5 ± 29.24* | (228.8 ± 33.07) | 97.0 ± 23.02 | (225.4 ± 41.79) |
| Alprenolol | 299.5 ± 12.19* | (453.6 ± 86.33) | 179.2 ± 53.05 | (290.9 ± 55.07) |
| Carvedilol | 86.3 ± 19.42 | (565.2 ± 192.8)* | 904.1 ± 43.46* | (1444.0 ± 202.0) |
| Nadolol | 181.9 ± 48.28 | (695.1 ± 286.3)* | 785.5 ± 154.8* | (1591.6 ± 335.0)* |

### Example 3

### Chronic Inverse Agonist Treatment Increases β-Adrenergnic Receptor Numbers as Monitored by Immunohistochemistry

For immunohistochemistry analysis of β₂-adrenergic receptor levels, non-drug-treated control mice and mice treated chronically with the β₂-adrenergic inverse agonist nadolol were used. The mice were sacrificed and the lungs excised. Then the lungs were fixed in 4% paraformaldehyde (45 min, 0° C.). After fixation, lungs were washed in PBS (60 min) and placed in increasing concentrations of sucrose (10% sucrose/5% glycine in PBS for 30 min; 20% sucrose/10% glycine in PBS for 30 min; 30% sucrose/25% glycine in PBS for 12 h at 4° C.). Lungs were embedded in OCT and 12-μm sections cut with a Tissue-Tek II cryostat. The sections were air dried and fixed with 4% paraformaldehyde for 15 min. After 3 washes in PBS, the slides were blocked with 5% milk in PBS for 1 h, and then incubated overnight with anti-β₂-adrenergic receptor antibody (1:200, Santa Cruz Biotechnology) in blocking solution. Slides were washed in PBS and incubated with secondary antibody (1:200, Cy3 goat anti-rabbit, 16 h at 4° C.). Control slides were incubated with antibody specific blocking peptide to demonstrate specificity of binding of the primary antibody. After washing with PBS, coverslips were mounted and viewed by epifluorescent microscopy.

As shown in FIG. 4, labeling with anti-β₂-adrenergic receptor antibodies was considerably more intense in lungs from animals treated with nadolol than in lungs from untreated animals (A, control+antibody; B, control+antibody+blocking peptide; C, nadolol+antibody; D, nadolol+antibody+blocking peptide). Loss of this signaling upon incubation in the presence of the β₂-adrenergic receptor peptide demonstrates that this antibody is specifically binding the β₂-adrenergic receptor. This observation is consistent with the radioligand binding data of Example 2 and suggests that β₂-adrenergic receptors are effectively upregulated by chronic administration of β₂-adrenergic inverse agonist drugs.

### Example 3

### Effect of Combination of Carvedilol and Salbutamol on Airway Hyperresponsiveness

The effect of combination therapy with carvedilol and salbutamol was compared to monotherapy with carvedilol alone on airway hyperresponsiveness in asthmatic mice.

Mice (Balb/cJ) aged 6 weeks were housed under specific pathogen-free conditions and fed a chicken ovalbumin-free diet. Mice were systemically sensitized with ovalbumin adsorbed to aluminum hydroxide. Mice were treated as follows: CAR/SAL 28D=for 28 days mice (n=6-12) were administered carvedilol (2400 ppm in animal chow) and salbutamol (subcutaneous delivery of 0.5 mg/kg/day in an Alzet #2400 osmotic minipump); NTX S/C=mice (n=6-12) no drug treatment for 28 days; CTRL=mice (n=6-12) no drug treatment for 28 days, not subsequently challenged; CARHD 28D=for 28 days mice (n=6-12) were administered carvedilol only (2400 ppm in animal chow); CARHD 28D SAL AC=for 28 days mice (n=6-12) were administered carvedilol (2400 ppm in animal chow) and 15 minutes prior to measuring airway hyperresponsiveness, salbutamol was administered at a dose of 1.2 mg.kg.

To measure airway hyperresponsiveness after 28 days, all mice except the CTRL (control) mice were challenged with ovalbumin and then all mice were anesthetized, tracheotomized, and connected to a Flexivent small animal ventilator to measure airway resistance (R_{aw}) by the forced oscillation technique. To induce airway constriction, a solution containing 150 μg/mL of methacholine was infused using a syringe infusion pump. The methacholine infusion was started at 0.008 mL/min and its rate was doubled stepwise up to a maximum 0.136 mL/min. Each methacholine dose was administered until a plateau was reached, during which data were sampled at 1-min intervals for 3-5 min and then averaged.

In FIG. 5A, at the highest dose of methacholine, both of the combination drug treatments were equally effective in preventing bronchoconstriction and not statistically significantly different from the control mice which were only challenged with saline solution. The carvedilol monotherapy resulted in higher bronchoconstriction than these treatments but less than the non-drug treated sensitized and challenged (NTX S/C) mice. Thus, the combination therapy of β₂-adrenergic inverse agonist and agonist with the agonist administered either chronically or acutely is effective at ameliorating airway hyperresponsiveness to allergen and methacholine challenge and is an improvement over the monotherapy of the β₂-adrenergic inverse agonist alone.

This data is summarized in FIG. 5B, which shows that the combination of carvedilol and salbutamol is the most effective in reducing airway hyperresponsiveness of the treatments for which the results are shown in FIG. 5A. This indicates the effectiveness of the use of combination therapy of β₂-adrenergic inverse agonist and agonist.

### Example 4

### Effect of Combination Therapy with Aminophylline on Acute Airway Effects of Nadolol

Mice were sensitized to the allergen ovalbumin as described in Example 1. Mice were then challenged with allergen and then subjected to methacholine-indiced bronchoconstriction challenge, non-drug treated, NTX S/C, or pretreated with nadolol at 0.72 mg/kg i.p. for 15 minutes prior to methacholine challenge (nadolol acute treatment).

At time point 1 (time=−10 min) baseline airway resistance of the mice was determined. At time point 2 (time=−5 min) methacholine was infused into mice to reach their EC₇₀. At time point 3 (time=0 min) aminophylline was administered i.p. at a dose of 100 mg/kg.

In FIG. 6, pretreatment of mice with nadolol resulted in the same baseline airway resistance as non-drug treated sensitized and allergen-challenged mice. However, upon methacholine challenge, the nadolol-treated mice exhibited a much higher airway resistance of ˜4.5 versus 2.5 units. Upon administration of aminophylline, there was a significant and sustained drop in airway resistance in both the untreated and the nadolol-treated mice.

Z. Callaerts-Vegh et al., 37 Effects of Acute and Chronic Administration of β-Adrenoceptor Ligands on Airway Function in a Murine Model of Asthma,” Proc. Natl. Acad. Sci. USA 101: 49484953 (2004), have shown that while nadolol administered chronically prevents airway hyperresponsiveness in the same mouse asthma model, nadolol administered acutely worsens airway hyperresponsiveness. These data demonstrate that the addition of the methylxanthine aminophylline can alleviate the acute effects on airway hyperresponsiveness of nadolol administration. This is beneficial in that the opportunity exists for asthma subjects to take nadolol chronically to prevent bronchoconstriction. These subjects then can co-administer a methylxanthine such as aminophylline to prevent the acute detrimental effects of nadolol.

### Example 5

### Effect of Treatment with Salbutamol or Nadolol on the Ratio of Phospholipase C to Actin in Cultured Tracheal Smooth Muscle Cells

Cultured tracheal smooth muscle cells were obtained from mice exposed to the following treatments: NS/NC=nonasthmatic, non-challenged mice; S/C=asthmatic mice; Sal.Ac=asthmatic mice, acute salbutamol treatment; Sal.Ch=asthmatic mice, chronic salbutamol treatment; Nad.Ac=asthmatic mice, acute nadolol high dose treatment; and Nad.Ch=asthmatic mice, chronic nadolol high dose treatment.

After airway function experiments, the trachea were surgically removed from anesthetized mice that had been treated with drugs or vehicle. The trachea was minced and the cells plated and grown in culture. The smooth muscle cells grow faster and take over the culture dish. The cells were grown in medium which contained the drugs used in the treatment or vehicle controls. Phospholipase C (PLC-β1) was determined by immunoblotting with an antibody specific for the enzyme. Actin was used as a loading control and the amount of PLC-β1 was expressed as a ratio to actin.

The phospholipase C protein level was measured in these cultured cells and compared with the level of the structural protein actin as a baseline. The enzyme phospholipase C plays a key role in the pathway leading to asthmatic symptoms, as it cleaves a phosphodiester bond in membrane phospholipids, resulting in the formation of a 1,2-diglyceride. Arachidonate is then released from the diglyceride by the sequential actions of diglyceride lipase and monoglyceride lipase. Once released, a portion of the arachidonate is metabolized rapidly, leading to oxygenated products, including eicosanoids such as prostaglandins. Thus, any treatment that can inhibit phospholipase C activity is relevant for the treatment of asthma and other conditions characterized by abnormal receptor signaling.

The results are shown in FIG. 7. The results shown in FIG. 7 indicate that chronic administration of nadolol significantly decreases the activity of phospholipase C. This indicates that such chronic administration of nadolol is effective against conditions characterized by hypofunction of GPCR and prevents activation of some of the mechanisms that lead to the symptoms of conditions characterized by hypofunction of GPCR.

### Example 6

### Effect of β-Adrenergic Receptor Drugs at Low and High Doses on Airway Resistance

For these experiments, salbutamol was used for chronic administration at 0.5 mg/kg/day with a minipump and for acute administration at 0.15 mg/kg by i.v. bolus 15 minutes prior to challenge. Alprenolol was used at a high dose of 7200 ppm in chow or at a low dose of 720 ppm in chow. Carvedilol was used at a high dose of 2400 ppm in chow or at a low dose of 720 ppm in chow. Nadolol was used at a high dose of 250 ppm in chow or at a low dose of 25 ppm in chow. Nadolol was also tested at 1 ppm in chow and these results were identical to the untreated mice.

The results are shown in FIGS. 8A (salbutamol); 8B (high-dose alprenolol); 8C (low-dose alprenolol); 8D (high-dose carvedilol); 8E (low-dose carvedilol); 8F (high-dose nadolol); and 8G (low-dose nadolol). In these diagrams, Ctrl=control mice, non-asthmatic, non-drug treated; NTX=asthmatic mice, non-drug treated; AC=acute administration; 2d=chronic administration for 2 days; 28d=chronic administration for 28 days. The airway resistance (R_{aw}) is plotted as cm H₂O ml⁻¹ s. The data particularly shows the effect of the β-adrenergic inverse agonists carvedilol and nadolol in providing protection from airway hyperresponsiveness with chronic administration.

### Example 7

### Correlation of Decrease in Airway Resistance with Upregulation of β-Adrenergic Receptor Density

The correlation of the decrease in airway resistance with the upregulation of β-adrenergic receptor density for three different periods of administration of salbutamol, alprenolol, carvedilol, and nadolol is shown in Table 2. The periods of administration of the agents are 15 minutes, 2 days, and 28 days. Only the inverse agonists carvedilol and nadolol showed an increase in β-adrenergic receptor density at periods longer than 15 minutes; carvedilol showed an increase in receptor density at 28 days, while nadolol showed an increase in receptor density at both 2 days and 28 days. There was an exact correlation between the decrease of airway resistance (R_{aw}) and the increase in receptor density. This strongly supports the concept of combination therapy, such as with an inverse agonist and an agonist.

TABLE 2
Correlation of Decrease In Airway Resistance With
Upregulation of β₂-Adrenergic Receptor Density

| | 15 minutes | | 2 days | | 28 days | |
|---|---|---|---|---|---|---|
| | | Increased | | Increased | | Increased |
| | Decreased R_{aw} | β₂AR density | Decreased R_{aw} | β₂AR density | Decreased R_{aw} | β₂AR density |
| Salbutamol | yes | yes | no | no | no | no |
| Alpreriolol | yes | yes | no | no | no | no |
| Carvedilol | no | no | no | no | yes | yes |
| Nadolol | no | no | yes | yes | yes | yes |

### Example 8

### Effects of Chronic Treatment with Metoprolol and Timolol on Airway Hyperresponsiveness in Asthmatic Mice

The protocols of Example 1 were followed for two additional inverse agonists, metoprolol (dosage of 20 mg/kg administered 3× daily via subcutaneous injection for 7 days) and timolol (dosage of 20 mg/kg in chow for 7 days), using asthmatic mice and methacholine challenge as in Example 1. Airway resistance (R_{aw}) was measured as in Example 1. The results for metoprolol and timolol are shown in FIG. 9A. The results were compared to historical controls as shown in FIG. 9B: Ctd, no drug treatment, no challenge with methacholine; NTX, no drug treatment, challenged with methacholine. The results indicate that chronic treatment with either metoprolol and timolol is effective in reducing airway hyperresponsiveness in asthmatic mice.

### ADVANTAGES OF THE INVENTION

The present invention provides a improved method of treating many conditions mediated by GPCR, and avoids the tolerance or tachyphylaxis that often is the consequence of conventional therapy with agonists directed to those receptors. The use of inverse agonists, in essence, forces the body to respond by improving its own signaling mechanisms to counter the disease or condition being treated. Accordingly, compositions and methods that employ inverse agonists have broad potential for treating such diseases and conditions without the induction of tolerance. This promises superior long-term results in the treatment of such conditions without interfering with short-term acute therapy.

The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein.

In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. The disclosures of all articles and references, including patent publications, are incorporated herein by reference.

1. A method for treating a disease or condition associated with the activity of a G protein coupled receptor (GPCR) comprising administering an inverse agonist for the GPCR to an organism with a disease or condition associated with the activity of the GPCR in a quantity and for a period that causes an increase in the population of GPCRs, either spontaneously active or those that are available and activated by an endogenous agonist or by an exogenous agonist, associated with that physiological function, thereby producing a therapeutic effect to ameliorate the disease or condition.

2. The method of claim 1 wherein the administration of the inverse agonist results in continuous levels of the inverse agonist in the bloodstream of the organism to which the inverse agonist is being administered.

3. The method of claim 1 wherein the disease or condition associated with the activity of a GPCR is a pulmonary airway disease.

4.-6. (canceled)

7. The method of claim 3 wherein the GPCR is a β₂-adrenergic receptor.

8. The method of claim 7 wherein the therapeutic effect is an upregulation of the population of pulmonary β₂-adrenergic receptors.

9. The method of claim 7 wherein the therapeutic effect is increased pulmonary airway relaxation responsiveness to β₂-adrenergic agonist drugs.

10. The method of claim 7 wherein the inverse agonist is selected from the group consisting of nadolol, bupranolol, butoxamine, carazolol, carvedilol, ICI-1 18,551, levobunolol, metoprolol, propranolol, sotalol, and timolol, and the salts, solvates, analogues, congeners, bioisosteres, hydrolysis products, metabolites, precursors, and prodrugs thereof.

11. The method of claim 10 wherein the β-adrenergic inverse agonist is selected from the group consisting of nadolol and a compound of formula (I) 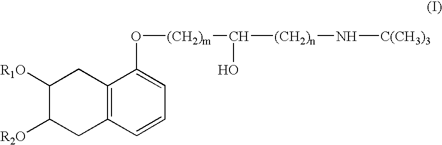
wherein R₁ is hydrogen or lower alkyl, R₂ is hydrogen or lower alkyl, and m and n are 1 to 3, with the proviso that where R₁ and R₂ are both hydrogen and m is 1, n is other than 1.

12. The method of claim 11 wherein the β-adrenergic inverse agonist is nadolol.

13. The method of claim 10 wherein the β-adrenergic inverse agonist is selected from the group consisting of carvedilol and a compound of formula (II). 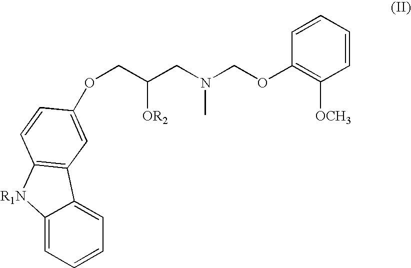
wherein R₁ is hydrogen or lower alkyl, R₂ is hydrogen or lower alkyl, and R₃ is hydrogen or lower alkyl, with the proviso that all of R₁, R₂, and R₃ are not all hydrogen.

14. The method of claim 13 wherein the β-adrenergic inverse agonist is carvedilol.

15.-18. (canceled)

19. The method of claim 3 wherein the method further comprises the administration of an additional agent.

20. The method of claim 19 wherein the additional agent is selected from the group consisting of a β₂-selective adrenergic agonist drug, a steroid, an anticholinergic drug, a xanthine compound, an anti-IgE antibody, a leukotriene modifier, and a phosphodiesterase IV inhibitor.

21.-36. (canceled)

37. The method of claim 1 wherein the disease or condition associated with the activity of a GPCR is congestive heart failure (CHF).

38. The method of claim 37 wherein the GPCR is a β₂-adrenergic receptor.

39. The method of claim 38 wherein the inverse agonist is nadolol.

40. The method of claim 1 wherein the GPCR is selected from the group consisting of acetylcholine receptors, β-adrenergic receptors, β₃-adrenergic receptors, serotonin (5-hydroxytryptamine) receptors, dopamine receptors, adenosine receptors, angiotensin Type II receptors, bradykinin receptors, calcitonin receptors, calcitonin gene-related receptors, cannabinoid receptors, cholecystokinin receptors, chemokine receptors, cytokine receptors, gastrin receptors, endothelin receptors, γ-aminobutyric acid (GABA) receptors, galanin receptors, glucagon receptors, glutamate receptors, luteinizing hormone receptors, choriogonadotrophin receptors, follicle-stimulating hormone receptors, thyroid-stimulating hormone receptors, gonadotrophin-releasing hormone receptors, leukotriene receptors, Neuropeptide Y receptors, opioid receptors, parathyroid hormone receptors, platelet activating factor receptors, prostanoid (prostaglandin) receptors, somatostatin receptors, thyrotropin-releasing hormone receptors, vasopressin and oxytocin receptors.

41. The method of claim 40 further comprising administering an agonist to the GPCR.

42. A method for screening a compound for inverse agonist activity against a GCPR comprising the steps of:
(a) providing a population of specific G protein coupled receptors characterized by a constitutive basal level of activity in the absence of an agonist;
(b) contacting the population of specific G protein coupled receptors with a compound to be screened for its inverse agonist activity, the compound not being an agonist of the population of specific G protein coupled receptors; and
(c) determining the constitutive basal level of activity of the specific G protein coupled receptors in the absence of the compound and in the presence of the compound, such that the constitutive basal level of activity decreases if the compound is an inverse agonist.

43.-47. (canceled)

48. The method of claim 42 wherein a physiological consequence of receptor activation is measured.

49. The method of claim 48 wherein the physiological consequence of receptor activation is airway resistance.

50.-51. (canceled)

52. A method for screening a compound for inverse agonist activity against a GCPR comprising the steps of:
(a) providing cells containing a population of specific G protein coupled receptors characterized by a constitutive basal level of activity in the absence of an agonist;
(b) contacting the cells containing the population of specific G protein coupled receptors with a compound to be screened for its inverse agonist activity, the compound not being an agonist of the population of specific G protein coupled receptors, the compound being contacted with the cells for a period of time to result in an increase in receptor population or receptor density if the compound is an inverse agonist; and
(c) determining the receptor population or receptor density of the specific G protein coupled receptors in the cells in the absence of the compound and in the presence of the compound, such that the receptor population or receptor density increases if the compound is an inverse agonist.

53.-54. (canceled)

55. A method for treating a disease or condition associated with the activity of a G protein coupled receptor (GPCR) comprising administering an inverse agonist for the GPCR to an organism with a disease or condition associated with the activity of the GPCR in a quantity and for a period that prevents the decrease in the population of GPCRs due to the presence of either exogenous or endogenous agonist, thereby producing a therapeutic effect to ameliorate the disease or condition.

56.-59. (canceled)

60. A pharmaceutical composition comprising:
(a) a therapeutically effective amount of an inverse agonist for a GPCR;
(b) a therapeutically effective amount of a second therapeutic agent, the second therapeutic agent being selected from the group consisting of a β₂-selective adrenergic agonist, a steroid, an anticholinergic drug, a xanthine compound, an anti-IgE antibody, a leukotriene modifier, and a phosphodiesterase IV inhibitor; and
(c) a pharmaceutically acceptable carrier.

61.-67. (canceled)
